(19) United States
(12) Patent Application Publication
(10) Pub. No.: US 2008/0274133 A1
(43) Pub. Date: Nov. 6, 2008

(54) Soluble Bifunctional Proteins

(75) Inventor: Bent Karsten Jakobsen, Oxfordshire (GB)

Correspondence Address: BANNER & WITCOFF, LTD., 1100 13th STREET, N.W., SUITE 1200, WASHINGTON, DC, 20005-4051, US

(73) Assignee: AVIDEX LTD., Abingdon, Oxfordshire (GB)

(21) Appl. No.: 11/667,276

(22) PCT Filed: Nov. 17, 2005
(86) PCT No.: PCT/GB05/04449

§ 371 Date: Jun. 16, 2008

(30) Foreign Application Priority Data
Nov. 18, 2004 (GB) 0425368.8
Feb. 9, 2005 (GB) 0502681.0

(51) Int. Cl. A61K 39/385; C12N 15/62; C07K 16/46; C12N 5/06; C12N 15/85; C12P 21/02; A61P 35/00

(52) U.S. Cl. 424/193.1; 536/23.5; 530/391.1; 435/375; 435/320.1; 435/325; 435/69.7

(57) ABSTRACT

The present invention provides a soluble bifunctional protein comprising an association between a T cell receptor and a superantigen. Also provided are therapeutic compositions comprising said bifunctional proteins and methods for the use thereof.

The present invention relates to soluble bifunctional proteins comprising an association between a T cell receptor (TCR) and a superantigen. Such proteins have value in the treatment of diseases such as cancer and infection.

### BACKGROUND TO THE INVENTION

### Soluble TCRs

A number of constructs have been devised to date for the production of soluble TCRs. These constructs fall into two broad classes, single-chain TCRs and dimeric TCRs, and the literature relevant to these constructs is summarised below.

### Single Chain TCRs

Single-chain TCRs (scTCRs) are artificial constructs consisting of a single amino acid strand, which like native heterodimeric TCRs bind to MHC-peptide complexes. Unfortunately, attempts to produce functional alpha/beta analogue scTCRs by simply linking the alpha and beta chains such that both are expressed in a single open reading frame have been unsuccessful, presumably because of the natural instability of the alpha-beta soluble domain pairing.

Accordingly, special techniques using various truncations of either or both of the alpha and beta chains have been necessary for the production of scTCRs. These formats appear to be applicable only to a very limited range of scTCR sequences. Soo Hoo et al (1992) PNAS. 89 (10): 4759-63 report the expression of a mouse TCR in single chain format from the 2C T cell clone using a truncated beta and alpha chain linked with a 25 amino acid linker and bacterial periplasmic expression (see also Schodin et al (1996) Mol. Immunol. 33 (9): 819-29). This design also forms the basis of the m6 single-chain TCR reported by Holler et al (2000) PNAS. 97 (10): 5387-92 which is derived from the 2C scTCR and binds to the same H2-Ld-restricted alloepitope. Shusta et al (2000) Nature Biotechnology 18: 754-759 report using single-chain 2 C TCR constructs in yeast display experiments, which produced mutated TCRs with, enhanced thermal stability and solubility. This report also demonstrated the ability of these displayed 2C TCRs to selectively bind cells expressing their cognate pMHC. Khandekar et al (1997) J. Biol. Chem. 272 (51): 32190-7 report a similar design for the murine D10 TCR, although this scTCR was fused to MBP and expressed in bacterial cytoplasm (see also Hare et al (1999) Nat. Struct. Biol. 6 (6): 574-81). Hilyard et al (1994) PNAS. 91 (19): 9057-61 report a human scTCR specific for influenza matrix protein-HLA-A2, using a Vα-linker-Vβ design and expressed in bacterial periplasm.

Chung et al (1994) PNAS. 91 (26) 12654-8 report the production of a human scTCR using a Vα-linker-Vβ-Cβ design and expression on the surface of a mammalian cell line. This report does not include any reference to peptide-HLA specific binding of the scTCR. Plaksin et al (1997) J. Immunol. 158 (5): 2218-27 report a similar Vα-linker-Vβ-Cβ design for producing a murine scTCR specific for an HIV gp120-H-2D^{d} epitope. This scTCR is expressed as bacterial inclusion bodies and refolded in vitro.

### Dimeric TCRs

A number of papers describe the production of TCR heterodimers which include the native disulfide bridge which connects the respective subunits (Garboczi, et al., (1996), Nature 384(6605): 134-41; Garboczi, et al., (1996), J Immunol 157(12): 5403-10; Chang et al., (1994), PNAS USA 91: 11408-11412; Davodeau et al., (1993), J. Biol. Chem. 268(21): 15455-15460; Golden et al., (1997), J. Imm. Meth. 206: 163-169; U.S. Pat. No. 6,080,840). However, although such TCRs can be recognised by TCR-specific antibodies, none were shown to recognise its native ligand at anything other than relatively high concentrations and/or were not stable.

In WO 99/60120, a soluble TCR is described which is correctly folded so that it is capable of recognising its native ligand, is stable over a period of time, and can be produced in reasonable quantities. This TCR comprises a TCR α or γ chain extracellular domain dimerised to a TCR β or δ chain extracellular domain respectively, by means of a pair of C-terminal dimerisation peptides, such as leucine zippers. This strategy of producing TCRs is generally applicable to all TCRs. Guillaume et al., (2003) Nature Immunology 4: 657-663, details the construction of a soluble JM22 TCR containing an introduced disulfide inter-chain bond between amino acids attached to the C terminus of the construct. This particular construct was derived from the extracellular portion of the JM22 TCR, truncated a single amino acid N terminal to the position of the native disulfide inter-chain bond. C terminal constant domain extensions were added to both the α and β chains of this TCR. These extensions caused the position of the inter-chain forming cysteine residues to be displaced downstream by three amino acids in the α chain and six amino acids in the β chain relative to their native positions. Soluble TCRs of this general design, that is soluble TCRs comprising introduced C terminal constant domain extensions containing a disulfide inter-chain disulfide bond, may also be used in multivalent TCR complexes of the present invention.

Reiter et al, Immunity, 1995, 2:281-287, details the construction of a soluble molecule comprising disulfide-stabilised TCR α and β variable domains, one of which is linked to a truncated form of Pseudomonas exotoxin (PE38). One of the stated reasons for producing this molecule was to overcome the inherent instability of single-chain TCRs. The position of the novel disulfide bond in the TCR variable domains was identified via homology with the variable domains of antibodies, into which these have previously been introduced (for example see Brinkmann, et al. (1993), Proc. Natl. Acad. Sci. USA 90: 7538-7542, and Reiter, et al. (1994) Biochemistry 33: 5451-5459). However, as there is no such homology between antibody and TCR constant domains, such a technique could not be employed to identify appropriate sites for new inter-chain disulfide bonds between TCR constant domains.

### Superantigens

Superantigens are bacterial or viral proteins which cause immunostimulation by cross-linking Class II MHC molecules on the surface of antigen presenting cells (APCs) to TCRs of a defined subset of a chain variable domains. This cross-linking causes polyclonal T cell activation leading to a massive release of cytokines such as IL-2 and TNF-β which can cause lethal toxic shock syndrome. (Li et al., (1999) Annu Rev Immunol 17 435-466) provides a review of the structure and function of superantigens.

### Superantigen Fusion Proteins

There are a number of publications that relate to the use of superantigen fusion proteins.

U.S. Pat. No. 6,692,746 provides methods for treating a subject having a tumor comprising administering to the subject a tumoricidally effective amount of a composition consisting of a biologically active homologue of a staphylococcal enterotoxin or a streptococcal pyrogenic exotoxin, or such toxins fused to a polypeptide fusion partner.

U.S. Pat. No. 6,514,498 discloses conjugates between target-seeking moieties and superantigens modified in one or more amino acid residues in a region determining binding to TCR and T cell activation.

U.S. Pat. No. 6,197,299 discloses soluble antibody conjugates comprising a superantigen covalently linked by peptide bond linkage to an antibody which is specific for a cell surface structure on a cell, preferably a cancer and methods of treatment using such conjugates.

EP0998305 discloses methods and compositions for causing the cytolysis of target cells using superantigen—targeting agent—immune-modulator conjugates which induce T-cell activation.

WO03094846 discloses methods for treating tumors comprising intratumoral administration of a superantigen or superantigen fusion proteins and/or intrathecal or intracavitary administration of a superantigen directly into the sheath.

(Ueno et al., (2002) Anticancer Res. 22 (2A) 769-76) details the use of a recombinant fusion protein of SEA and the single-chain variable fragment (scFv) of the FU-MK-1 antibody, which recognises a glycoprotein antigen (termed MK-1 antigen) present on carcinomas. This study concluded that this fusion protein may serve as a potentially useful immunotherapeutic reagent for human MK-1-expressing tumours.

Another study (Takemura et al., (2002) Cancer Immunol Immunother. 51 (1):33-44) detailed the bacterial production of a fusion protein comprising a mutated staphylococcal enterotoxin A (SEA) and an anti-MUC1/anti-CD3 diabody (Mx3 diabody). This fusion protein showed MUC1-specific antitumour effects in bile duct carcinoma (BDC)-xenografted severe combined immunodeficient (SCID) mouse models.

Finally, another study (Nielsen et al., (2000) J Immunother 23 (1): 146-53) detailed a Phase I study of single, escalating doses of a superantigen-antibody fusion protein (PNU-214565) in patients with advanced colorectal or pancreatic carcinoma. The fusion protein comprised staphylococcal enterotoxin A (SEA) and recombinant fusion of the Fab fragment of the monoclonal antibody C242 which recognises human colorectal (CRC) and pancreatic carcinomas (PC). The authors concluded that a single 3-hour infusion of PNU-214565 could be safely administered up to 4 ng/kg.

### BRIEF DESCRIPTION OF THE INVENTION

This invention makes available for the first time a soluble bifunctional protein comprising an association between a T cell receptor (TCR) and a superantigen.

### DETAILED DESCRIPTION OF THE INVENTION

The present invention provides a soluble bifunctional protein comprising an association between (a) a T cell receptor (TCR) and (b) a superantigen or a functional variant thereof.

There are a number of soluble TCR constructs suitable for use in the present invention. These include, but are not limited to, the following;

Single-chain TCRs (scTCRs) of the Vα-linker-Vβ design (Hilyard et al (1994) PNAS. 91 (19): 9057-61) or the Vα-linker-Vβ-Cβ design (Chung et al (1994) PNAS. 91 (26) 12654-8).

Dimeric TCRs (dTCRs) as described in WO 99/60120, Guillaume et al., (2003) Nature Immunology 4: 657-663, or Reiter et al, Immunity, 1995, 2:281-287.

One aspect of the invention is provided by a soluble bifunctional protein comprising an association between a T cell receptor (TCR), which comprises all or part of a TCR α chain except the transmembrane domain thereof and all or part of a TCR β chain except the transmembrane domain thereof, wherein each TCR chain comprise a functional variable domain and at least a part of the constant domain of the TCR chain and are linked by a disulfide bond between constant domain residues which is not present in native TCR, and a superantigen or a functional variant thereof.

Superantigens are bacterial or viral proteins which cause immunostimulation by cross-linking Class II MHC molecules on the surface of antigen presenting cells (APCs) to TCRs of a defined subset of β chain variable domains. As used herein the term “functional variant” of a superantigen refers to analogues of a given superantigen which function in the same way, resulting in immunostimulation. For example, as is known to those skilled in the art, it functional variants can incorporate minor changes in their amino acid sequence compared to a given wild-type or mutant. Such minor changes include conservative amino acid substitutions, single amino acid deletions, and truncations remote from the Class II MHC and TCR β chain binding domains. Functional variants will normally have a sequence identity with the parent superantigen of at least 75%, more often at least 90% and in most case at least 95-99%.

The bifunctional proteins of the present invention are soluble. One test of solubility is the ability of the bifunctional proteins to exist as a mono-disperse species in phosphate buffered saline (PBS) (KCL 2.7 mM, KH₂PO₄ 1.5 mM, NaCl 137 mM and Na₂PO₄ 8 mM, pH 7.1-7.5. Life Technologies, Gibco BRL) at a concentration of 1 mg/ml, and for >90% of said bifunctional proteins to remain as a mono disperse species after incubation at 25° C. for 1 hour. For example, to assess the solubility of the bifunctional protein, it is first purified as described in Example 3. Following this purification, 100 μg of the bifunctional protein is analysed by analytical size exclusion chromatography e.g. using a Pharmacia Superdex 75 HR column equilibrated in PBS. A further 100 μg of the bifunctional protein is incubated at 25° C. for 1 hour and then analysed by size exclusion chromatography as before. The size exclusion traces are then analysed by integration and the areas under the peaks corresponding to the mono disperse species are compared. The relevant peaks may be identified by comparison with the elution position of protein standards of known molecular weight. The mono-disperse heterodimeric soluble bifunctional protein has a molecular weight of approximately 75-80 kDa.

As used herein the term “association” refers to a linkage between the TCR and the superantigen, which forces the TCR and superantigen into close proximity such that the two behave biologically as a single entity. Such linkage will normally be covalent. Consequently, the term “bifunctional protein” as used herein is to be understood as including:
A fusion protein comprising the TCR and superantigen sequences
The TCR and superantigen sequences covalently linked by a non-peptidic linker
A complex in which the TCR and superantigen sequences are associated non-covalently.

Where the superantigen is indirectly to the TCR via a linker radical, there are two classes of linker radical that are preferred for the association of TCRs and superantigens of the present invention, non-peptidic polymeric radicals and peptidic radicals. These two classes of linker radicals are discussed in detail below in relation to their use in the formation of multivalent complexes of the soluble bifunctional proteins of the invention. Example 1 herein provides examples of peptidic linkers which may be used to form the association between the TCR and superantigens.

In certain embodiments of the invention, the N-terminus of the superantigen is covalently linked directly, or indirectly via a linker radical, to the C-terminal amino acid of the TCR α chain or TCR β chain. In further embodiments of the invention the N-terminus of the superantigen is either (a) directly linked to the C-terminal amino acid of the TCR α chain or TCR β chain via a peptide bond, or inter cysteine disulfide bond, or (b) linked indirectly via a peptide bond, or inter cysteine disulfide bond, to the C-terminal amino acid of a linker amino acid sequence which is itself linked via a peptide bond, or inter cysteine disulfide bond, to the C-terminus of the TCR α chain or TCR β chain.

In a specific embodiment of the invention N-terminus of the superantigen is linked to the C-terminal amino acid of the TCR α chain or TCR β chain via a peptidic linker.

As is known to those skilled in the art a variety of peptide linkers may be suitable to link the TCR β chains to the superantigens. The following are examples linker sequences which may be used for this purpose

ggcggtccg which encodes a Gly-Gly-Pro linker (L1).
ggatccggcggtccg (SEQ ID NO: 1)—which encodes a Gly-Ser-Gly-Gly-Pro (SEQ ID NO: 2) linker (L2) including a BamH1 restriction enzyme site.
ggatccggtgggggcggaagtggaggcagcggtggatccggcggtccg—(SEQ ID NO: 3) which encodes a Gly-Ser-Gly-Gly-Gly-Gly-Ser-Gly-Gly-Ser-Gly-Gly-Ser-Gly-Gly-Pro (SEQ ID NO: 4) linker (L3) including two BamH1 restriction enzyme sites.
cccggg—which encodes a Pro-Gly linker (L4) including a Xma1 restriction enzyme site

In a specific embodiment of the invention N-terminus of the superantigen is linked to the C-terminal amino acid of the TCR α chain or TCR β chain via a non-peptidic polymeric radical.

One aspect of the invention is provided wherein the superantigen is a mutant of a wild type superantigen, wherein the mutation reduces the affinity of the superantigen for Class II MHC molecules whilst retaining the affinity for TCR 13 variable domains.

In a further aspect of the invention the superantigen is a wild type or mutated staphylococcal superantigen.

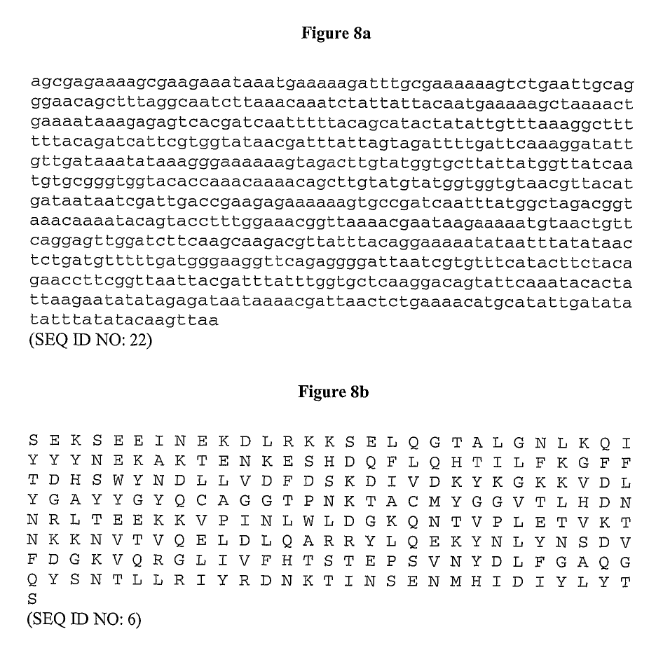

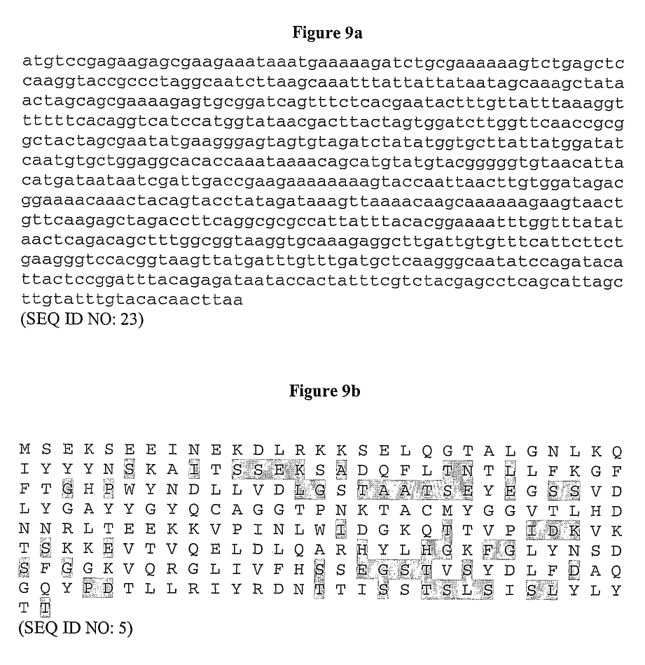

A specific embodiment of the invention is provided wherein the superantigen has the amino acid sequence shown in FIG. 9b (SEQ ID NO: 5). This superantigen, (SEA-E120, Active Biotech, Sweden) has been mutated relative to the wild-type SEA-E superantigen shown in FIG. 8b (SEQ ID NO: 6) to reduce the affinity of the superantigen for Class II MHC molecules whilst retaining the affinity for TCR β variable domains.

The TCR parts of the soluble bifunctional proteins disclosed herein are targeting moieties. They target TCR ligands such as peptide-MHC or CD1-antigen complexes. As such, it would be desirable if these TCR had a higher affinity and/or a slower off-rate for the TCR ligand than native TCRs specific for that ligand.

In one broad aspect, the TCR parts of the soluble bifunctional proteins of the invention are in the form of either single chain TCRs (scTCRs) or dimeric TCRs (dTCRs) as described in WO 04/033685 and WO 03/020763 respectively. In particular:

Suitable TCR parts of the soluble bifunctional proteins comprises all or part of a TCR α chain except the transmembrane domain thereof and all or part of a TCR β chain except the transmembrane domain thereof, wherein each TCR chain comprise a functional variable domain and at least a part of the constant domain of the TCR chain and are linked by a disulfide bond between constant domain residues which is not present in native TCR.

In one specific embodiment of the invention such TCR parts comprise all of the extracellular constant Ig domain of the TCR chain.

In another specific embodiment of the invention such TCR parts comprise all of the extracellular domain of the TCR chain.

The TCR parts of the soluble bifunctional proteins are characterised by having a disulfide link between constant domain residues which is not present in native TCR.

In one aspect of the invention this covalent disulfide bond links a residue of the immunoglobulin region of the constant domain of the α chain to a residue of the immunoglobulin region of the constant domain of the β chain.

Another aspect of the invention is provided wherein, in the TCR part, an interchain disulfide bond present in native TCRs is absent. A specific embodiment of this aspect provided wherein, in the TCR part, native α and β TCR chains are truncated at the C-terminus such that the cysteine residues which form the native interchain disulfide bond are excluded. In an alternative embodiment the cysteine residues which form the native interchain disulfide bond are substituted to another residue. In another specific embodiment, the cysteine residues which form the native interchain disulfide bond are substituted to serine or alanine.

One aspect of the invention is provided wherein, in the TCR part, an unpaired cysteine residue present in native TCR β chains is not present

Another aspect of the present invention is provided wherein, in the TCR part, the disulfide bond which is not present in native TCRs is between cysteine residues substituted for residues whose β carbon atoms are less than 0.6 nm apart in the native TCR structure.

A specific embodiment of the invention is provided wherein, in the TCR part, the disulfide bond which is not present in native TCRs is between cysteine residues substituted for Thr 48 of exon 1 of TRAC*01 and Ser 57 of exon 1 of TRBC1*01 or TRBC2*01.

Another specific embodiment of the invention is provided wherein, in the TCR part, the disulfide bond which is not present in native TCRs is between cysteine residues substituted for Thr 45 of exon 1 of TRAC*01 and Ser 77 of exon 1 of TRBC1*01 or TRBC2*01.

Another specific embodiment of the invention is provided wherein, in the TCR part, the disulfide bond which is not present in native TCRs is between cysteine residues substituted for Tyr 10 of exon 1 of TRAC*01 and Ser 17 of exon 1 of TRBC1*01 or TRBC2*01.

Another specific embodiment of the invention is provided wherein, in the TCR part, the disulfide bond which is not present in native TCRs is between cysteine residues substituted for Thr 45 of exon 1 of TRAC*01 and Asp 59 of exon 1 of TRBC1*01 or TRBC2*01.

Another specific embodiment of the invention is provided wherein, in the TCR part, the disulfide bond which is not present in native TCRs is between cysteine residues substituted for Ser 15 of exon 1 of TRAC*01 and Glu 15 of exon 1 of TRBC1*01 or TRBC2*01.

Another specific embodiment of the invention is provided by a soluble bifunctional protein comprising an association between:

(a) A TCR, which comprises (i) all or part of a TCR α chain, except the transmembrane domain thereof, and (ii) all or part of a TCR β chain, except the transmembrane domain thereof, wherein (i) and (ii) each comprise a functional variable domain and at least a part of the constant domain of the TCR chain, and are linked by a disulfide bond between cysteine residues substituted for Thr 48 of exon 1 of TRAC*01 and Ser 57 of exon 1 of TRBC1*01 or TRBC2*01, and;
(b) The superantigen of SEQ ID NO: 5
wherein the association TCR (a) and the superantigen (b) are associated in C-terminal to N-terminal relationship respectively.

A further aspect of the invention is provided wherein, the TCR part comprises all or part of a TCR α chain except the transmembrane domain thereof and all or part of a TCR β chain except the transmembrane domain thereof, wherein each TCR chain each comprise the functional variable domain of a first TCR fused to all or part of the constant domain of a second TCR, the first and second TCRs being from the same species.

An additional aspect is provided wherein a soluble bifunctional protein of the invention further comprises a detectable label.

A further aspect is provided by a soluble bifunctional protein of the invention, wherein the TCR has an affinity (Kd) for a given peptide-MHC of higher than 1 μM. The inventors co-ending application WO 2004/044004 details methods of producing TCR having a higher affinity and/or a slower off-rate for the TCR ligand than native TCRs specific for that ligand. Preferably, the affinity (K_{D}) of the TCR for the TCR ligand is higher than 1 μM, and/or the off-rate (k_{OFF}) is slower than 1×10⁻³ s⁻¹. More preferably, the affinity (K_{D}) of the TCR for the TCR ligand is higher than 10 nM, and/or the off-rate (k_{off}) is slower than 1×10⁻⁴S⁻¹. Most preferably, the affinity (K_{D}) of the TCR for the TCR ligand is higher than 1 nM, and/or the off-rate (k_{off}) is slower than 1×10⁻⁵ S⁻¹.

The affinity (K_{D}) and/or off-rate (k_{off}) measurement can be made by any of the known methods. A preferred method is the Surface Plasmon Resonance (Biacore) method of Example 4.

In addition to the non-native disulfide bond referred to above, the TCR parts of the soluble bifunctional proteins of the invention may include a disulfide bond between residues corresponding to those linked by a disulfide bond in native TCRs.

The TCR parts of the soluble bifunctional proteins of the invention preferably do not contain a sequence corresponding to transmembrane or cytoplasmic sequences of native TCRs.

### Multivalent Complexes

One aspect of the invention provides a multivalent complex comprising a plurality of soluble bifunctional proteins. One embodiment of this aspect is provided by two or three or four associated bifunctional proteins associated with one another via a linker radical comprising a polyalkylene glycol polymer or a peptidic sequence. Preferably the complexes are water soluble, so the linker radical should be selected accordingly. Furthermore, it is preferable that the linker radical should be capable of attachment to defined positions on the soluble bifunctional proteins, so that the structural diversity of the complexes formed is minimised. One embodiment of the present aspect is provided by a multivalent complex of the invention wherein the polymer chain or peptidic linker sequence extends between amino acid residues of each soluble bifunctional protein which are not located in a variable region sequence of the TCR part thereof.

Since the complexes of the invention may be for use in medicine, the linker moieties should be chosen with due regard to their pharmaceutical suitability, for example their immunogenicity.

Examples of linker moieties which fulfil the above desirable criteria are known in the art, for example the art of linking antibody fragments.

There are two classes of linker that are preferred for use in the production of multivalent complexes of the present invention. A multivalent complex of the invention in which the soluble bifunctional proteins are linked by a polyalkylene glycol chain provides one embodiment of the present aspect.

The first are hydrophilic polymers such as polyalkylene glycols. The most commonly used of this class are based on polyethylene glycol or PEG, the structure of which is shown below.

HOCH₂CH₂O(CH₂CH₂O)ₙ—CH₂CH₂OH

Wherein n is greater than two. However, others are based on other suitable, optionally substituted, polyalkylene glycols include polypropylene glycol, and copolymers of ethylene glycol and propylene glycol.

Such polymers may be used to treat or conjugate therapeutic agents, particularly polypeptide or protein therapeutics, to achieve beneficial changes to the PK profile of the therapeutic, for example reduced renal clearance, improved plasma half-life, reduced immunogenicity, and improved solubility. Such improvements in the PK profile of the PEG-therapeutic conjugate are believe to result from the PEG molecule or molecules forming a ‘shell’ around the therapeutic which sterically hinders the reaction with the immune system and reduces proteolytic degradation. (Casey et al, (2000) Tumor Targeting 4 235-244) The size of the hydrophilic polymer used my in particular be selected on the basis of the intended therapeutic use of the TCR complex. Thus for example, where the product is intended to leave the circulation and penetrate tissue, for example for use in the treatment of a tumour, it may be advantageous to use low molecular weight polymers in the order of 5 KDa. There are numerous review papers and books that detail the use of PEG and similar molecules in pharmaceutical formulations. For example, see Harris (1992) Polyethylene Glycol Chemistry—Biotechnical and Biomedical Applications, Plenum, New York, N.Y. or Harris & Zalipsky (1997) Chemistry and Biological Applications of Polyethylene Glycol ACS Books, Washington, D.C.

The polymer used can have a linear or branched conformation. Branched PEG molecules, or derivatives thereof, can be induced by the addition of branching moieties including glycerol and glycerol oligomers, pentaerythritol, sorbitol and lysine.

Usually, the polymer will have a chemically reactive group or groups in its structure, for example at one or both termini, and/or on branches from the backbone, to enable the polymer to link to target sites in the soluble bifunctional protein. This chemically reactive group or groups may be attached directly to the hydrophilic polymer, or there may be a spacer group/moiety between the hydrophilic polymer and the reactive chemistry as shown below:
Reactive chemistry-Hydrophilic polymer-Reactive chemistry
Reactive chemistry-Spacer-Hydrophilic polymer-Spacer-Reactive chemistry

The spacer used in the formation of constructs of the type outlined above may be any organic moiety that is a non-reactive, chemically stable, chain, Such spacers include, by are not limited to the following:

—(CH₂)ₙ— wherein n=2 to 5

—(CH₂)₃NHCO(CH₂)₂

A multivalent complex of the invention in which a divalent alkylene spacer radical is located between the polyalkylene glycol chain and its point of attachment to a soluble bifunctional protein of the complex provides a further embodiment of the present aspect.

A multivalent complex of the invention in which the polyalkylene glycol chain comprises at least two polyethylene glycol repeating units provides a further embodiment of the present aspect.

There are a number of commercial suppliers of hydrophilic polymers linked, directly or via a spacer, to reactive chemistries that may be of use in the present invention. These suppliers include Nektar Therapeutics (CA, USA), NOF Corporation (Japan), Sunbio (South Korea) and Enzon Pharmaceuticals (NJ, USA).

Commercially available hydrophilic polymers linked, directly or via a spacer, to reactive chemistries that may be of use in the present invention include, but are not limited to, the following:

| PEG linker | | Catalogue |
|---|---|---|
| Description | Source of PEG | Number |
| Dimer linkers | | |
| 3.4K linear (Maleimide) | Nektar | 2D2DOFO2 |
| 5K forked (Maleimide) | Nektar | 2D2DOHOF |
| 10K linear (with orthopyridyl ds- | Sunbio | |
| linkers in place of Maleimide) | | |
| 20K forked (Maleimide) | Nektar | 2D2DOPOF |
| 20K linear (Maleimide) | NOF Corporation | |
| 40K forked (Maleimide) | Nektar | 2D3XOTOF |
| Higher order multimer linkers | | |
| 15K, 3 arms, Mal₃ (for trimer) | Nektar | OJOONO3 |
| 20K, 4 arms, Mal₄ (for tetramer) | Nektar | OJOOPO4 |
| 40K, 8 arms, Mal₈ (for octamer) | Nektar | OJOOTO8 |

A variety of coupling chemistries can be used to couple polymer molecules to protein and peptide therapeutics. The choice of the most appropriate coupling chemistry is largely dependent on the desired coupling site. For example, the following coupling chemistries have been used attached to one or more of the termini of PEG molecules (Source: Nektar Molecular Engineering Catalogue 2003):
N-maleimide
Vinyl sulfone
Benzotriazole carbonate
Succinimidyl proprionate
Succinimidyl butanoate
Thio-ester
Acetaldehydes
Acrylates
Biotin
Primary amines

As stated above non-PEG based polymers also provide suitable linkers for multimerising the soluble bifunctional proteins of the present invention. For example, moieties containing maleimide termini linked by aliphatic chains such as BMH and BMOE (Pierce, products Nos. 22330 and 22323) can be used.

Peptidic linkers are the other preferred class of linker radicals. These linkers are comprised of chains of amino acids, and function to produce simple linkers or multimerisation domains onto which soluble bifunctional proteins can be attached. The biotin/streptavidin system has previously been used to produce tetramers of soluble TCRs (see WO/99/60119) for in-vitro binding studies. However, strepavidin is a microbially-derived polypeptide and as such not ideally suited to use in a therapeutic.

A multivalent complex of the invention in which the soluble bifunctional proteins are linked by a peptidic linker derived from a human multimerisation domain provides a further embodiment of the present aspect.

There are a number of human proteins that contain a multimerisation domain that could be used in the production of such multivalent complexes. For example the tetramerisation domain of p53 which has been utilised to produce tetramers of scFv antibody fragments which exhibited increased serum persistence and significantly reduced off-rate compared to the monomeric scFV fragment. (Willuda et al. (2001) J. Biol. Chem. 276 (17) 14385-14392) Haemoglobin also has a tetramerisation domain that could potentially be used for this kind of application.

A multivalent complex comprising at least two soluble bifunctional proteins wherein at least one of said soluble bifunctional proteins is a soluble bifunctional protein of the invention provides another embodiment of this aspect.

The above aspect and embodiments thereof details methods of producing multivalent complex of the invention of the following form:

(AB)n

wherein A is the TCR part and B is the superantigen part of the soluble bifunctional protein. It is envisaged that alternative construct in which the TCR part and/or the superantigen part of the soluble bifunctional proteins are multimerised may be beneficial. These constructs may be of any of the following forms:

A(B)n

wherein a single TCR of the invention is linked to at least two superantigens, or

(A)nB

wherein at least two TCRs of the invention are linked to a single superantigen, or

(A)n(B)n

wherein at least two TCRs of the invention are linked to at least two superantigens

### Therapeutic Use

The invention also provides a method for delivering a superantigen to a target cell, which method comprises contacting potential target cells with a soluble bifunctional protein or multivalent complex in accordance with the invention under conditions to allow attachment of the soluble bifunctional protein or multivalent complex to the target cell, said soluble bifunctional protein or multivalent complex being specific for a given peptide-MHC complex.

In particular, the soluble bifunctional protein or multivalent complex of the present invention can be used to deliver superantigens to the location of cells presenting a particular antigen. This is useful in many situations, for example, against tumours or sites of infectious disease. A superantigen can be delivered such that it exercises its effect locally but not only on the cell to which it binds.

Thus, one particular strategy uses bifunctional proteins or multivalent complexes according to the invention specific for tumour antigens. For cancer treatment, the localisation in the vicinity of tumours or metastasis enhances the effect of the superantigen. Alternatively, the bifunctional protein or multivalent complex of the present invention can be used to deliver superantigens to the location of cells presenting a particular antigen related to an infectious disease.

Administration of an interferon (IFN), such as IFN-γ, to a patient prior to, and/or simultaneously with, the administration of the bifunctional protein or multivalent complex can increase levels of peptide-MHC expression on the target cells. This has particular benefit in the treatment of cancer.

Further embodiments of the invention are provided by a pharmaceutical composition comprising a bifunctional protein or multivalent complex of the invention together with a pharmaceutically acceptable carrier.

The invention also provides a method of treatment of cancer comprising administering to a subject suffering such cancer disease an effective amount of a bifunctional protein or multivalent complex of the invention. In a related embodiment, the invention provides for the use of a bifunctional protein or multivalent complex of the invention, in the preparation of a composition for the treatment of cancer. SEA E120 or a functional variant or fragment thereof, is a particularly preferred superantigen for use in the bifunctional protein or multivalent complex of the invention in the treatment of cancer.

The invention also provides a method of treatment of infectious disease comprising administering to a subject suffering such an infectious disease an effective amount of a bifunctional protein or multivalent complex of the invention. In a related embodiment the invention provides for the use of a bifunctional protein or multivalent complex of the invention, in the preparation of a composition for the treatment of infectious disease. SEA E120, or a functional variant or fragment thereof is a preferred superantigen for use in the bifunctional protein or multivalent complex of the invention in the treatment of infectious disease.

Cancers for treatment by the compositions and methods of the present invention include, but are not limited to; leukaemia, head and neck, lung, breast, colon, cervical, liver, pancreatic, ovarian, prostate, colon, liver, bladder, oesophageal, stomach, melanoma and testicular.

Infectious diseases for treatment by the compositions and methods of the present invention are those caused by intracellular infectious organisms. The term “intracellular infectious organisms” as used herein is understood to encompass any organisms capable of entering a human cell. Such organisms may cause disease directly, or directly leading to altered cell function. These organisms can be any of the following:

### Bacteria, Fungi, Viruses, Protozoa and Mycobacteria.

Examples of these diseases and the intracellular infectious organisms which cause them include, but are not limited to bubonic plague caused by the Yersinia pestis bacteria and T-cell leukemia cause by the HTLV-1 virus.

Therapeutic bifunctional proteins or multivalent complexes in accordance with the invention will usually be supplied as part of a sterile, pharmaceutical composition which will normally include a pharmaceutically acceptable carrier. This pharmaceutical composition may be in any suitable form, (depending upon the desired method of administering it to a patient). It may be provided in unit dosage form, will generally be provided in a sealed container and may be provided as part of a kit. Such a kit would normally (although not necessarily) include instructions for use. It may include a plurality of said unit dosage forms.

The pharmaceutical composition may be adapted for administration by any appropriate route, for example parenteral, transdermal or via inhalation, preferably a parenteral (including subcutaneous, intramuscular, or, most preferably intravenous) route. Such compositions may be prepared by any method known in the art of pharmacy, for example by mixing the active ingredient with the carrier(s) or excipient(s) under sterile conditions.

Dosages of the substances of the present invention can vary between wide limits, depending upon the disease or disorder to be treated, the age and condition of the individual to be treated, etc. and a physician will ultimately determine appropriate dosages to be used.

### Additional Aspects

A bifunctional protein or multivalent complex of the present invention may be provided in substantially pure form, or as a purified or isolated preparation. For example, it may be provided in a form which is substantially free of other proteins.

Also provided is a nucleic acid molecule comprising a first nucleic acid sequence encoding a superantigen fused to a second nucleic acid encoding a soluble TCR β chain. A related embodiment is provided by a nucleic acid molecule comprising a first nucleic acid sequence encoding a superantigen fused to a second nucleic acid encoding all or part of a TCR β chain except the transmembrane domain thereof, wherein the nucleic acid sequence encoding the TCR β chain comprises an introduced cysteine codon capable of forming a non-native disulfide bond between the constant domain residues of the encoded TCR β chain and a TCR α chain containing a further non-native cysteine residue.

Further provided is a nucleic acid molecule comprising a first nucleic acid sequence encoding a superantigen fused to a second nucleic acid encoding a soluble TCR α chain. A related embodiment is provided by a nucleic acid molecule comprising a first nucleic acid sequence encoding a superantigen fused to a second nucleic acid encoding all or part of a TCR α chain except the transmembrane domain thereof, wherein the nucleic acid sequence encoding the TCR α chain comprises an introduced cysteine codon capable of forming a non-native disulfide bond between the constant domain residues of the encoded TCR α chain and a TCR β chain containing a further non-native cysteine residue.

Also provided is a vector comprising a nucleic acid molecule or molecules of the invention and a host cell comprising such a vector

Also provided is a method for obtaining a soluble bifunctional protein, which method comprises:
incubating a host cell which comprises a vector comprising a nucleic acid molecule encoding a TCR β chain fused to a superantigen and a host cell which comprises a vector comprising a nucleic acid molecule encoding a TCR α chain under conditions causing expression of the respective TCR β chain-superantigen fusion and TCR α chain;
purifying the respective TCR β chain-superantigen fusion and TCR α chain; and
mixing the respective TCR β chain-superantigen fusion and TCR α chain under refolding conditions such that a covalent disulfide bond links a residue of the immunoglobulin region of the constant domain of the TCR α chain to a residue of the immunoglobulin region of the constant domain of the TCR β chain-superantigen fusion.

Further provided is an alternative method for obtaining a soluble bifunctional protein, which method comprises:
incubating a host cell which comprises a vector comprising a nucleic acid molecule encoding a TCR α chain fused to a superantigen and a host cell which comprises a vector comprising a nucleic acid molecule encoding a TCR β chain under conditions causing expression of the respective TCR α chain-superantigen fusion and TCR β chain;
purifying the respective TCR α chain-superantigen fusion and TCR β chain; and
mixing the respective TCR α chain-superantigen fusion and TCR β chain under refolding conditions such that a covalent disulfide bond links a residue of the immunoglobulin region of the constant domain of the TCR β chain to a residue of the immunoglobulin region of the constant domain of the TCR α chain-superantigen fusion.

Also provided is a method for enriching a diverse population of T cells for T cells presenting a given sub-set of TCR β chain variable domains, which comprises:
(i) providing a soluble bifunctional protein or a multivalent complex of the invention wherein the superantigen part thereof selectively binds to said given sub-set of TCR β chain variable domains;
(ii) contacting the soluble bifunctional protein or multivalent complex with antigen presenting cells (APCs) presenting Class II MHC-peptide complexes, and said diverse population of T cells; and
(iii) incubating the admixture of (ii) under conditions suitable for the formation of APC-soluble bifunctional protein-T cell complexes.

Preferred features of each aspect of the invention are as for each of the other aspects mutatis mutandis. The prior art documents mentioned herein are incorporated to the fullest extent permitted by law.

### EXAMPLES

The invention is further described in the following examples, which do not limit the scope of the invention in any way.

Reference is made in the following to the accompanying drawings in which:

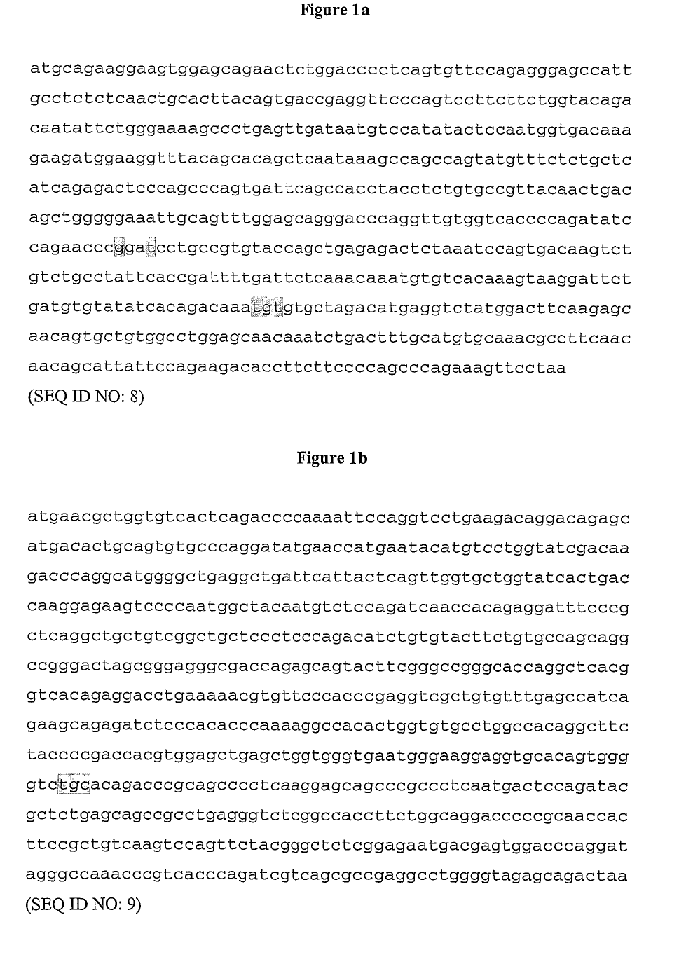

FIGS. 1a and 1b show respectively the nucleic acid sequences of the cc and P chains of a soluble A6 (Tax) TCR, mutated so as to introduce a cysteine codon. The shading indicates the introduced cysteine codons and an introduced BamH1 restriction site in the α chain nucleic acid;

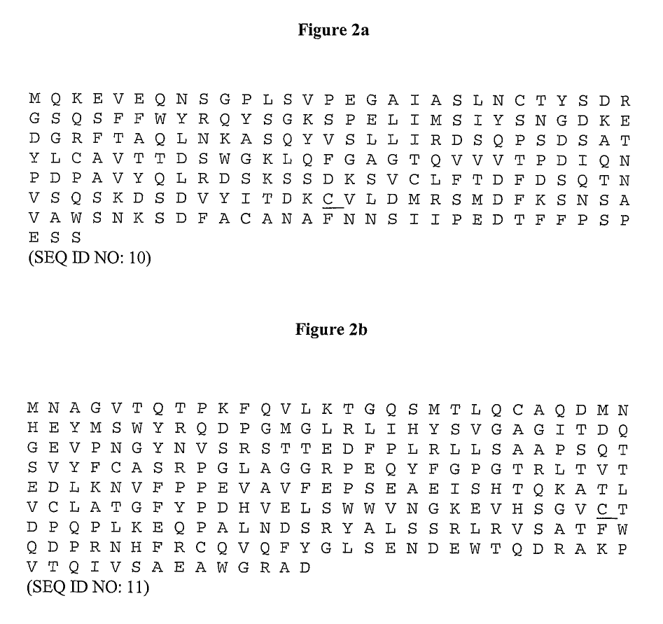

FIG. 2a shows the A6 (Tax) TCR α chain extracellular amino acid sequence, including the T₄₈→C mutation (underlined) used to produce the novel disulfide interchain bond, and FIG. 2b shows the A6 (Tax) TCR β chain extracellular amino acid sequence, including the S₅₇→C mutation (underlined) used to produce the novel disulfide inter-chain bond;

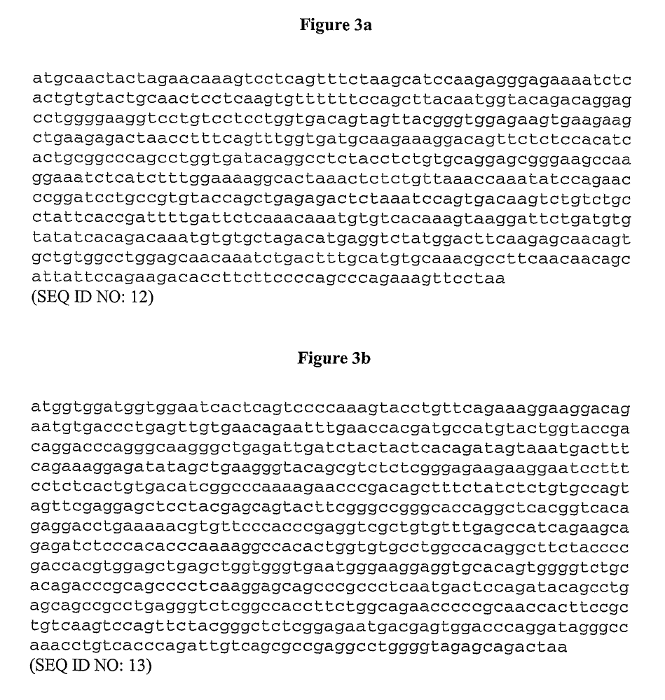

FIGS. 3a and 3b show the DNA sequence of α and β chain of the JM22 TCR mutated to include additional cysteine residues to form a non-native disulfide bond;

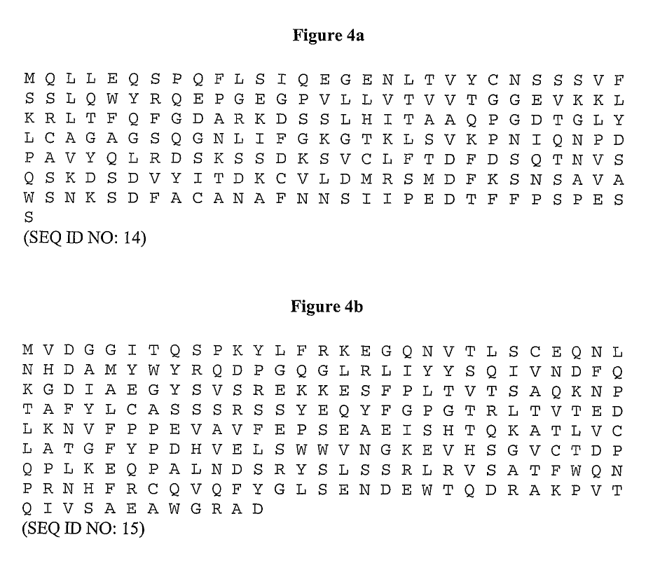

FIGS. 4a and 4b show respectively the JM22 TCR α and β chain extracellular amino acid sequences produced from the DNA sequences of FIGS. 3a and 3b;

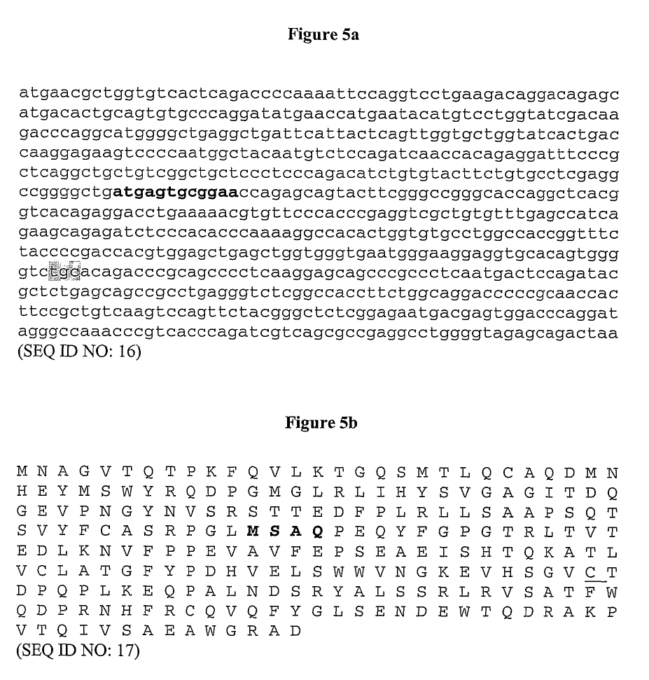

FIGS. 5a and 5b show the DNA and amino acid sequences of a high affinity variant of the A6 (Tax) TCR β chain mutated to include additional cysteine residues to form a non-native disulfide bond, the introduced cysteine codon is indicated by shading and the affinity increasing mutations are in bold;

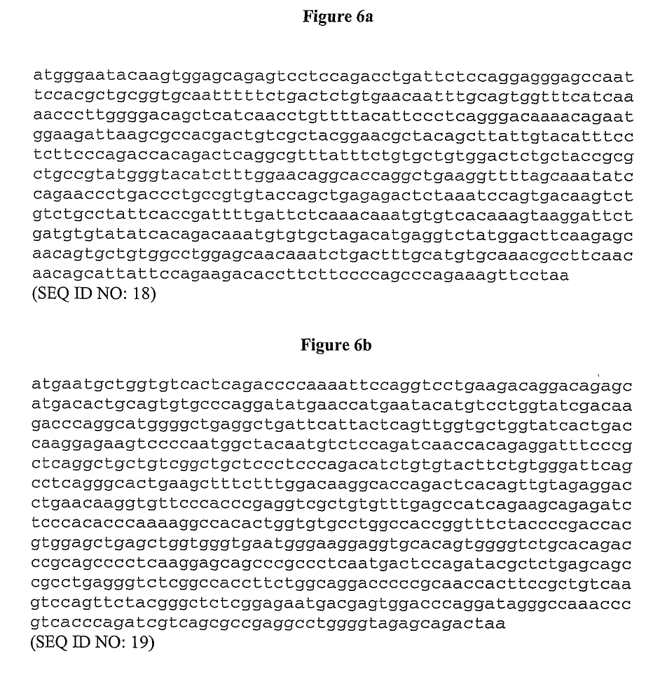

FIGS. 6a and 6b show the DNA sequence of α and β chain of a high affinity variant of a Telomerase TCR mutated to include additional cysteine residues to form a non-native disulfide bond, the introduced cysteine is indicated by shading;

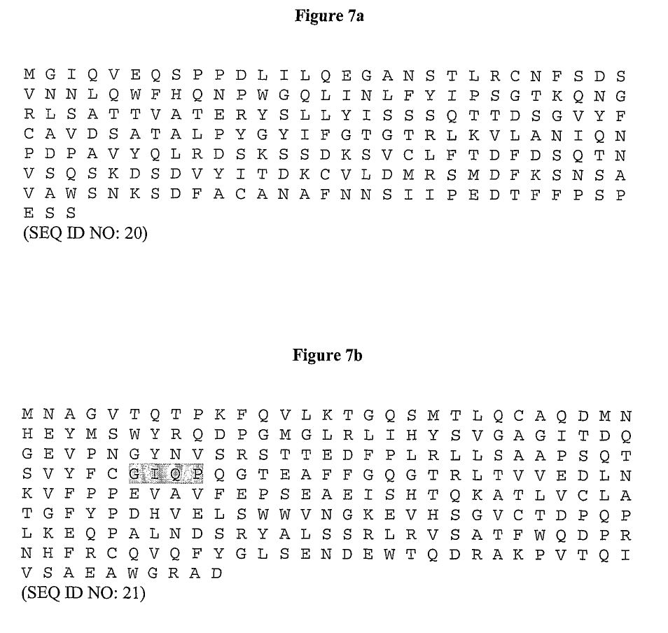

FIGS. 7a and 7b show respectively the high affinity variant of a Telomerase TCR α and β chain extracellular amino acid sequences produced from the DNA sequences of FIGS. 6a and 6b;

FIG. 8a—DNA sequence of wild-type SEA-E

FIG. 8b—Amino acid sequence of wild-type SEA-E

FIG. 9a—DNA sequence of the mutant superantigen SEA-E120

FIG. 9b—Amino acid sequence of the mutant superantigen SEA-E120, the mutated amino acids are indicated by shading

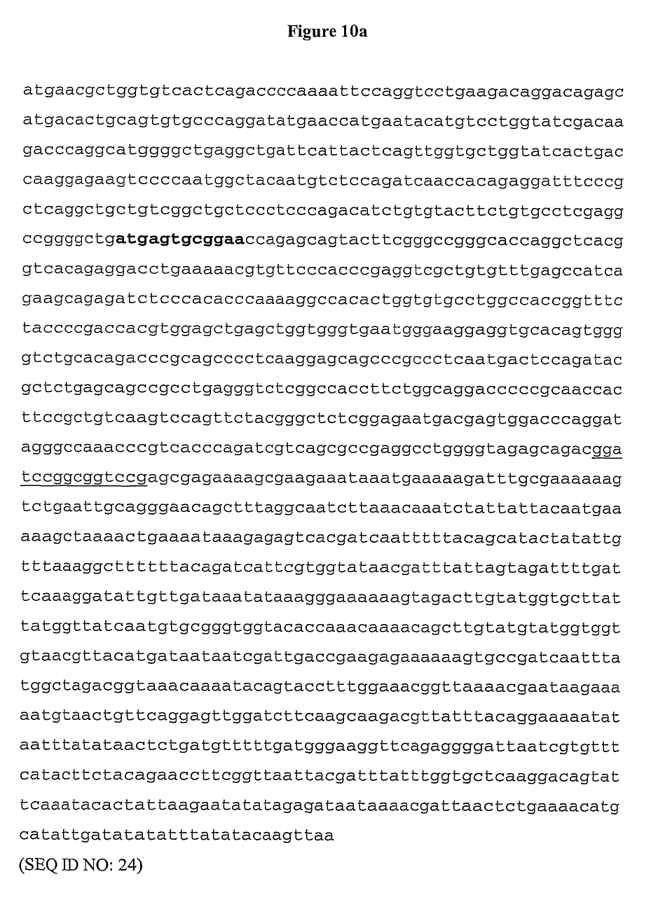

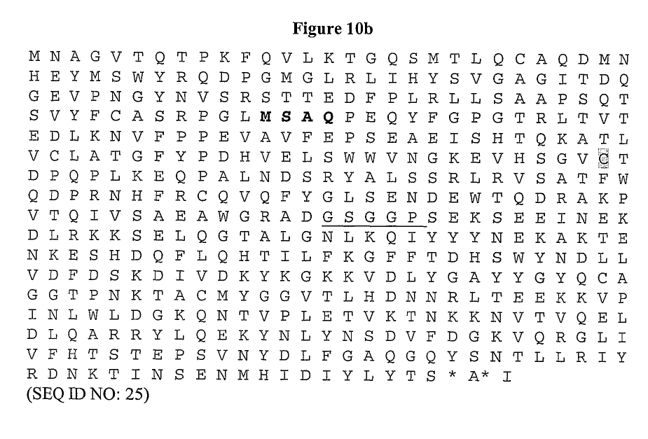

FIG. 10a—DNA sequence of the high affinity variant of the A6 (Tax) TCR β chain extracellular amino acid sequences containing a non-native cysteine involved in the formation of a novel interchain bond linked to the wild-type SEA E superantigen via a Gly-Ser-Gly-Gly-Pro (SEQ ID NO: 2) linker. The introduced cysteine is indicated by shading. The DNA sequence encoding the Gly-Ser-Gly-Gly-Pro linker is underlined.

FIG. 10b—Amino acid sequence of the high affinity variant of the A6 (Tax) TCR β chain extracellular amino acid sequences containing a non-native cysteine codon involved in the formation of a novel interchain bond linked to the wild-type SEA E superantigen via a Gly-Ser-Gly-Gly-Pro (SEQ ID NO: 2) linker. The introduced cysteine is indicated by shading. The Gly-Ser-Gly-Gly-Pro linker is underlined.

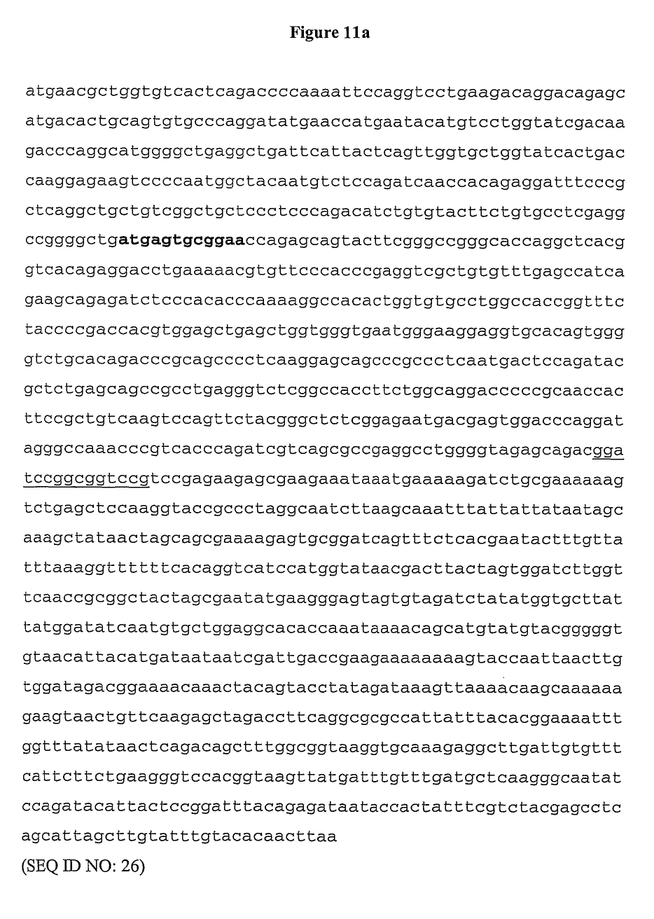

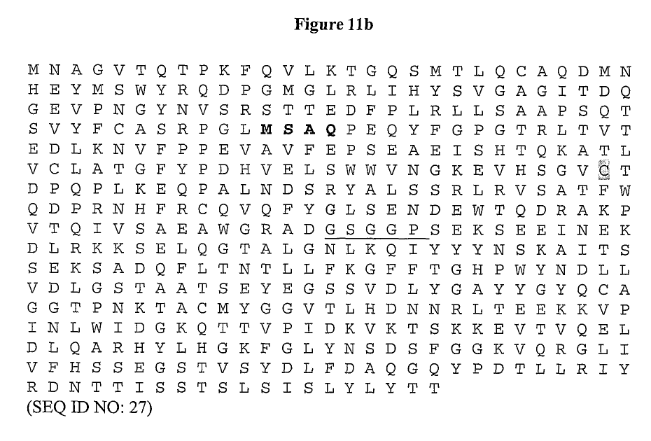

FIG. 11a—DNA sequence of the high affinity variant of the A6 (Tax) TCR β chain extracellular amino acid sequences containing a non-native cysteine involved in the formation of a novel interchain bond linked to the SEA E120 superantigen via a Gly-Ser-Gly-Gly-Pro (SEQ ID NO: 2) linker. The introduced cysteine is indicated by shading. The DNA sequence encoding the Gly-Ser-Gly-Gly-Pro linker is underlined.

FIG. 11b—Amino acid sequence of the high affinity variant of the A6 (Tax) TCR β chain extracellular amino acid sequences containing a non-native cysteine codon involved in the formation of a novel interchain bond linked to the SEA E120 superantigen via a Gly-Ser-Gly-Gly-Pro (SEQ ID NO: 2) linker. The introduced cysteine is indicated by shading. The Gly-Ser-Gly-Gly-Pro linker is underlined.

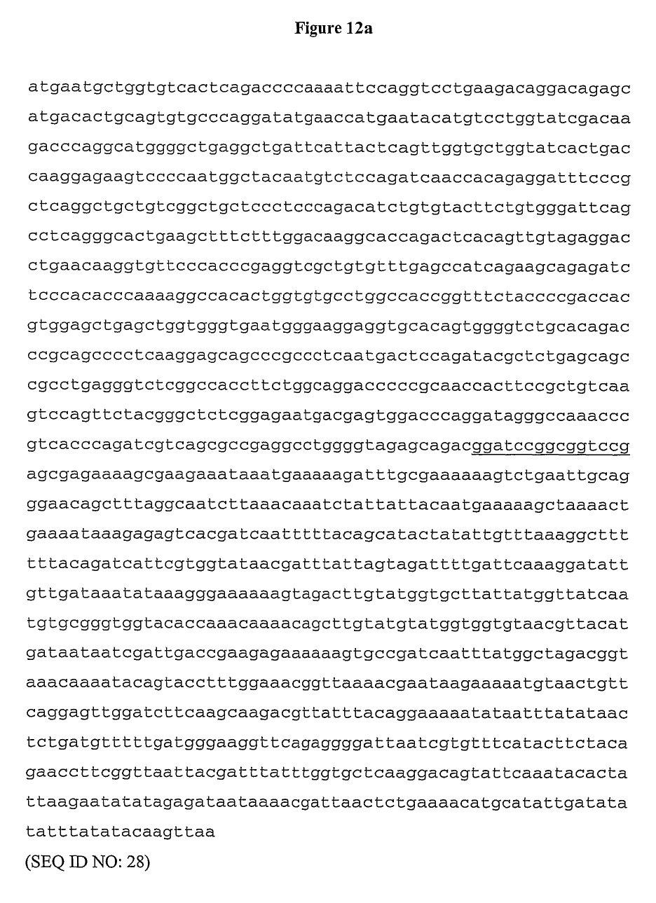

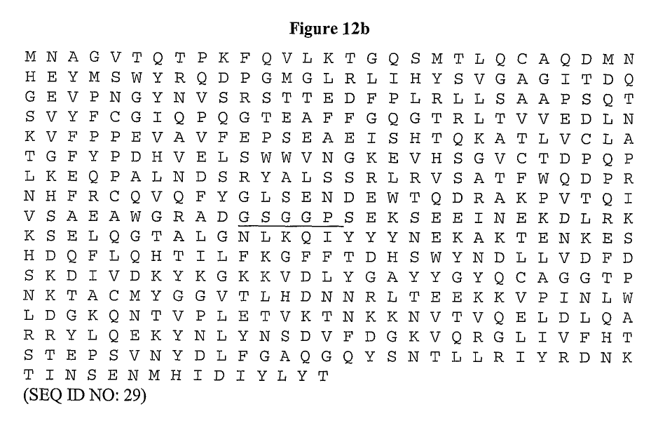

FIG. 12a—DNA sequence of a high affinity variant of a Telomerase TCR β chain extracellular amino acid sequences containing a non-native cysteine involved in the formation of a novel interchain bond linked to the wild-type SEA E superantigen via a Gly-Ser-Gly-Gly-Pro (SEQ ID NO: 2) linker. The introduced cysteine is indicated by shading. The DNA sequence encoding the Gly-Ser-Gly-Gly-Pro linker is underlined.

FIG. 12b—Amino acid sequence of a high affinity variant of a Telomerase TCR β chain extracellular amino acid sequences containing a non-native cysteine codon involved in the formation of a novel interchain bond linked to the wild-type SEA E superantigen via a Gly-Ser-Gly-Gly-Pro (SEQ ID NO: 2) linker. The introduced cysteine is indicated by shading. The Gly-Ser-Gly-Gly-Pro linker is underlined.

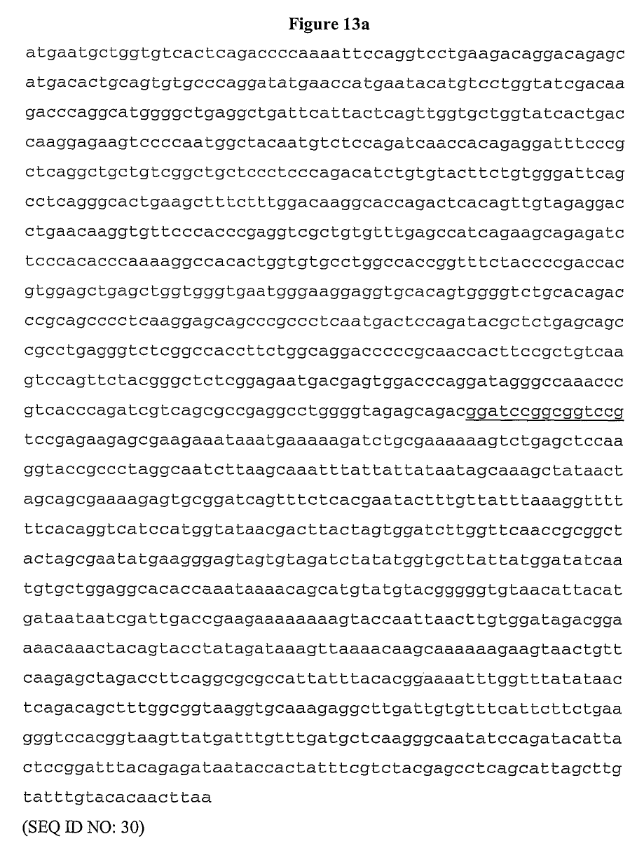

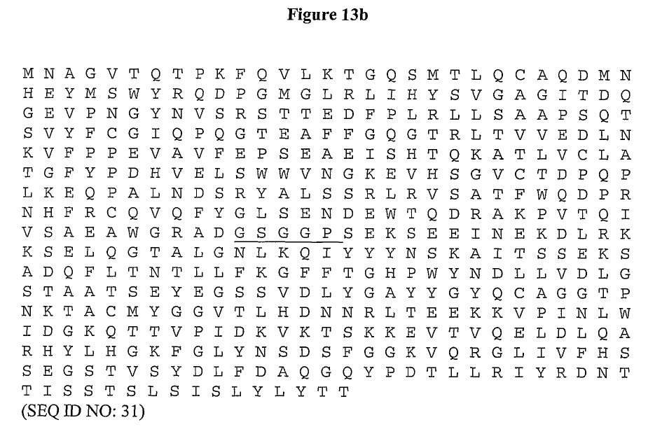

FIG. 13a—DNA sequence of a high affinity variant of a Telomerase TCR β chain extracellular amino acid sequences containing a non-native cysteine involved in the formation of a novel interchain bond linked to the SEA E120 superantigen via a Gly-Ser-Gly-Gly-Pro (SEQ ID NO: 2) linker. The introduced cysteine is indicated by shading. The DNA sequence encoding the Gly-Ser-Gly-Gly-Pro linker is underlined.

FIG. 13b—Amino acid sequence of a high affinity variant of a Telomerase TCR β chain extracellular amino acid sequences containing a non-native cysteine codon involved in the formation of a novel interchain bond linked to the SEA E120 superantigen via a Gly-Ser-Gly-Gly-Pro (SEQ ID NO: 2) linker. The introduced cysteine is indicated by shading. The Gly-Ser-Gly-Gly-Pro linker is underlined.

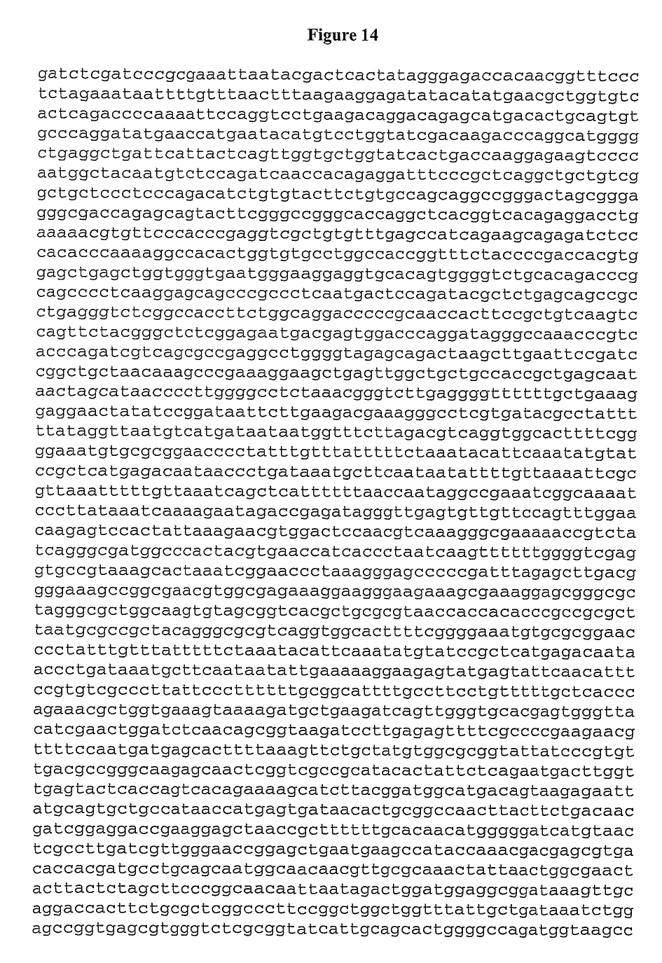

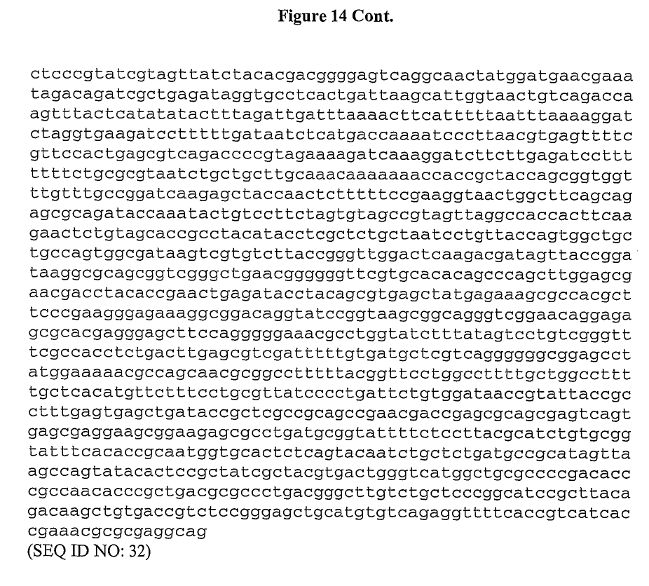

FIG. 14 details the DNA sequence of the pEX821 plasmid

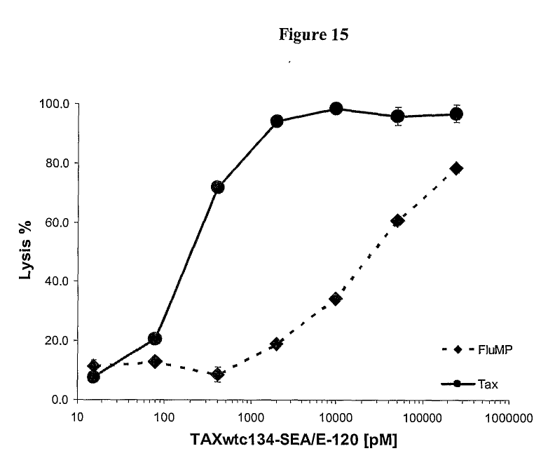

FIG. 15 shows peptide-MHC-specific, CTL-mediated killing of APCs using a high affinity A6 (Tax) TCR-SEA-E120 superantigen fusion protein.

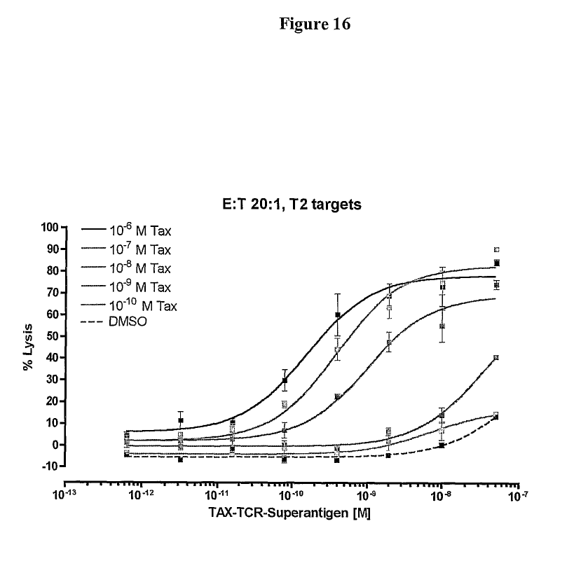

FIG. 16 shows the effect of titrating Tax peptide pulsing concentration (1×10⁻¹⁰ to 1×10⁻⁶M) and high affinity Tax TCR-L2-SEA E-120 concentration (6.4×10⁻¹³ to 1×10⁻⁷M) on the killing of T2 cells.

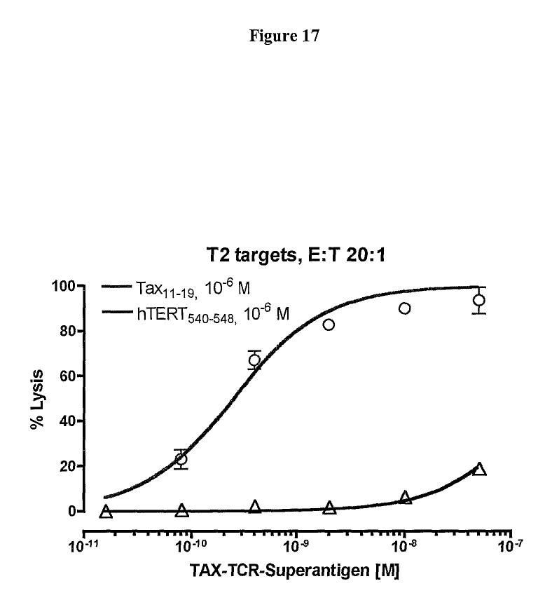

FIG. 17 shows the T2 cell killing elicited by a high affinity Tax TCR-L2-SEA E-120 fusion protein using a fixed concentration (10⁻⁶M) of Tax peptide or an irrelevant Telomerase peptide (ILAKFLHWL) control.

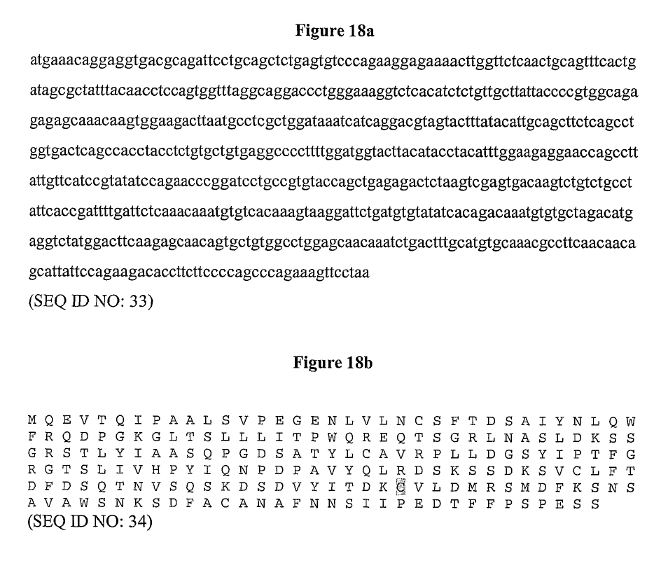

FIGS. 18a and 18b detail the DNA and amino acid sequence of a high affinity NY-ESO TCR α chain containing an introduced cysteine respectively

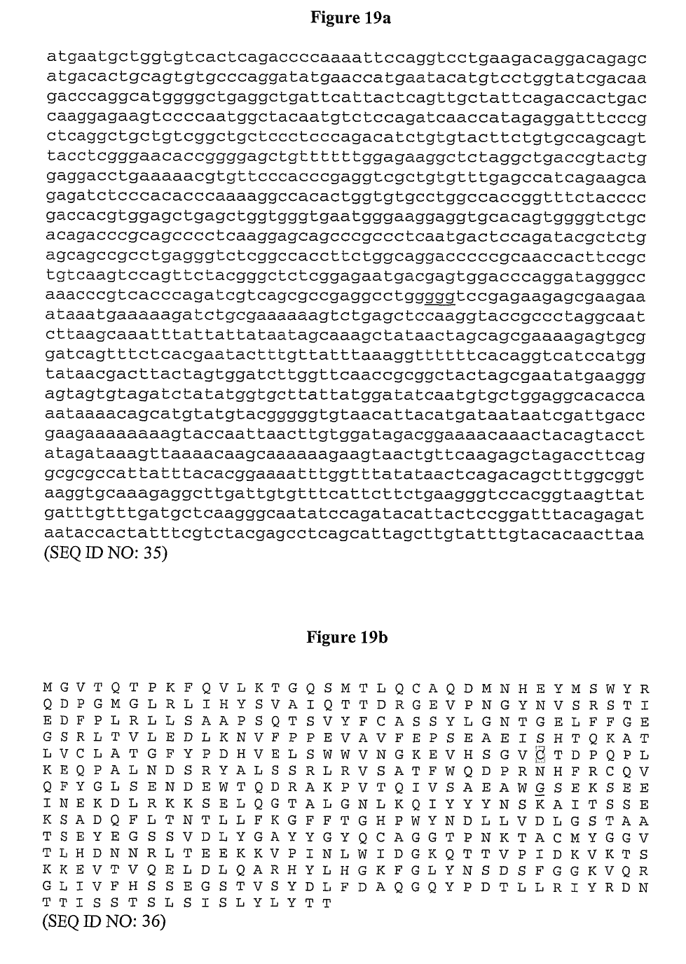

FIGS. 19a and 19b respectively detail the DNA and amino acid sequence of a truncated high affinity NY-ESO TCR β chain containing an introduced cysteine fused to SEA E-120 is detailed in FIGS. 19a and 19b respectively. The truncated NY-ESO TCR β chain has the last 3 amino acids (RAD) removed from the C-Terminal thereof compared to the “normal” soluble TCR. The codon encoding the final glycine residue in the DNA sequence of the truncated TCR β chain is underlined in FIG. 19a, as is the corresponding glycine residue in FIG. 19b.

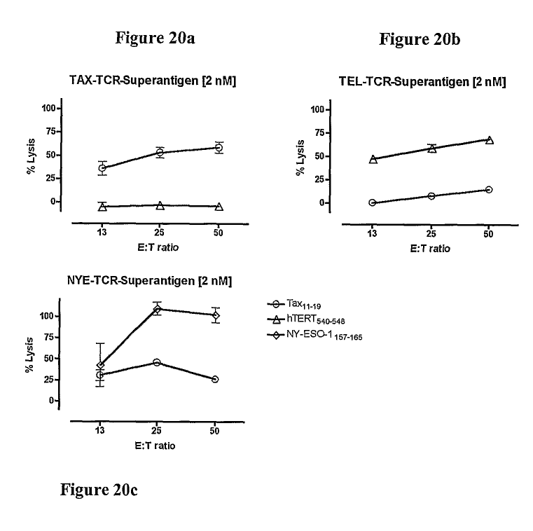

FIG. 20a to 20c show the T2 cell killing ability of a number of TCR-superantigen fusions, Tax TCR-L2-SEA E120, Telomerase TCR-L1-SEA E120 and NY-ESO TCR-LM1-SEA E120 respectively. All fusion s proteins were supplied at a concentration of 2 nM. The T2 target cells were pulsed with 110-6M cognate or irrelevant peptide. A range of Effector:Target cell (E:T) ratios were used.

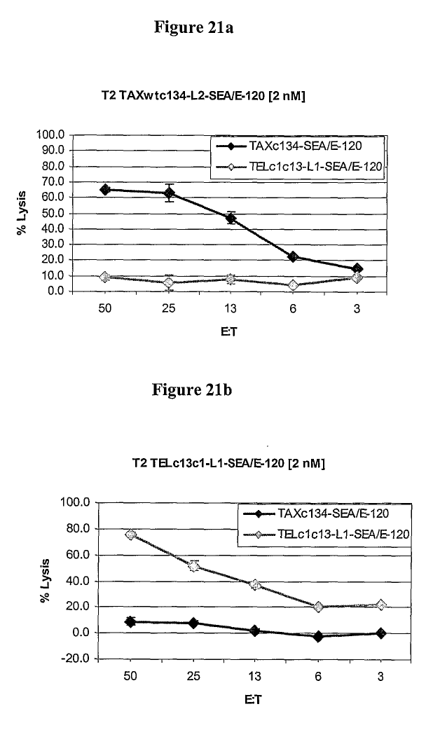

FIG. 21a and 21b show the T2 target cell killing ability of two different TCR-superantigen fusions (Tax TCR-L2-SEA E120 and Telomerase TCR-L1-SEA E120 respectively) both supplied at a concentration of 2 nM. The target cells were pulsed with 10⁻⁶M of the cognate peptide for one of these TCR-superantigen fusions and a range of Effector:Target cell (E:T) ratios were used.

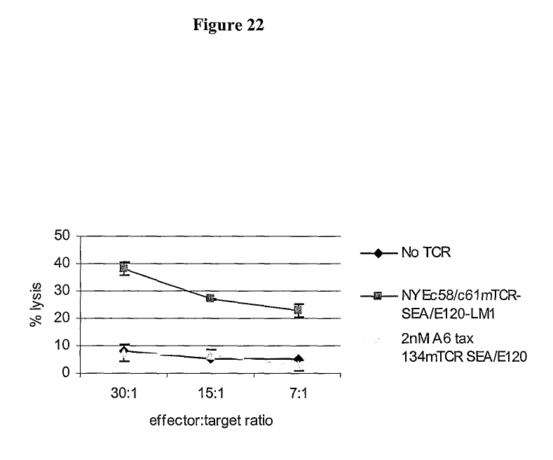

FIG. 22 shows the SK-MeI 37 tumour cell line killing ability of the Tax TCR-L2-SEA E120 and Telomerase TCR-L1-SEA E120 fusion proteins both supplied at a concentration of 2 nM. The target cells were pulsed with 10⁻⁶M of Tax peptide and a range of Effector:Target (E:T) cell ratios were investigated.

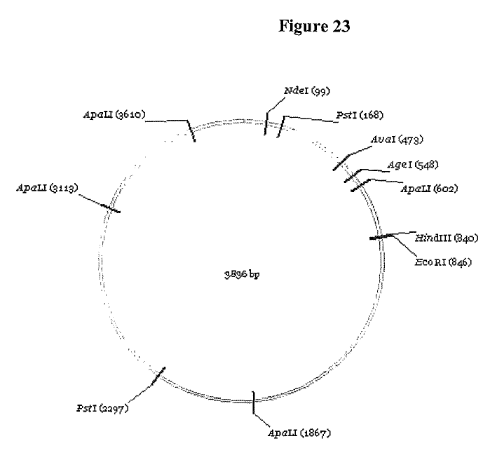

FIG. 23 provides the plasmid map of the pEX821 vector

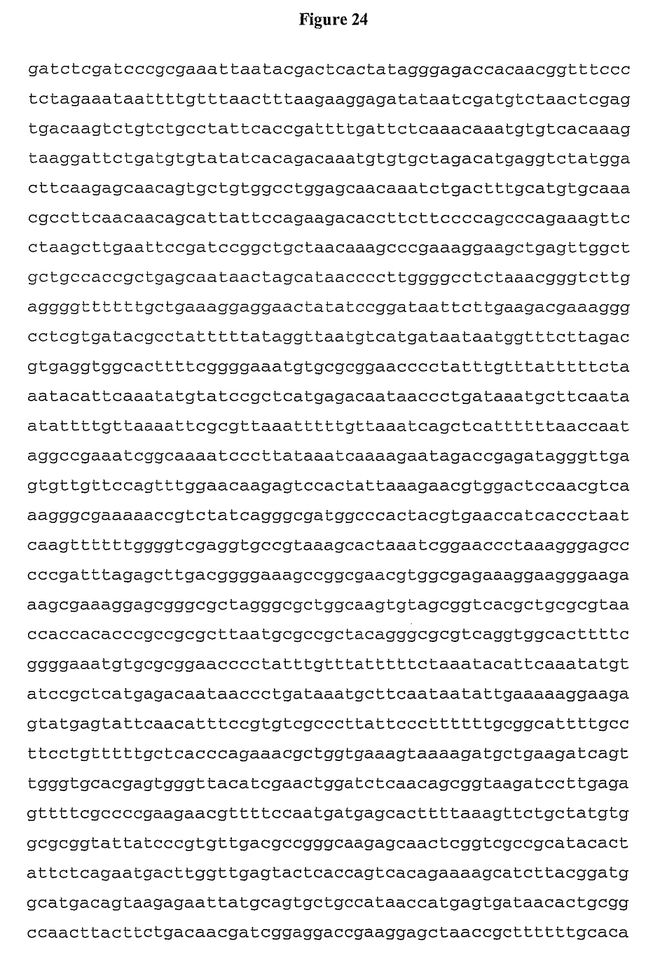

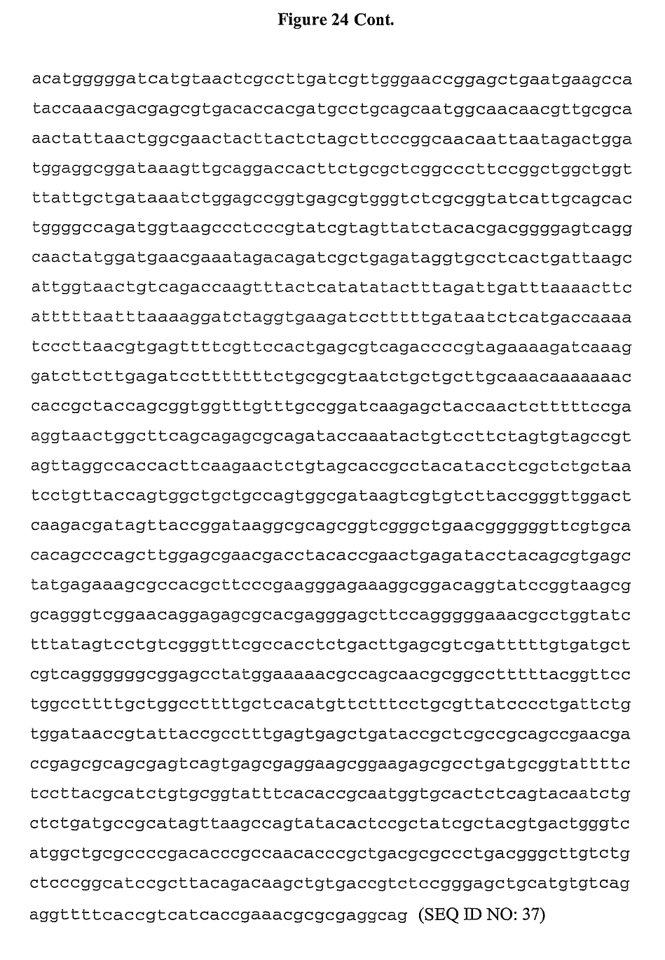

FIG. 24 details the DNA sequence of the pEX954 vector

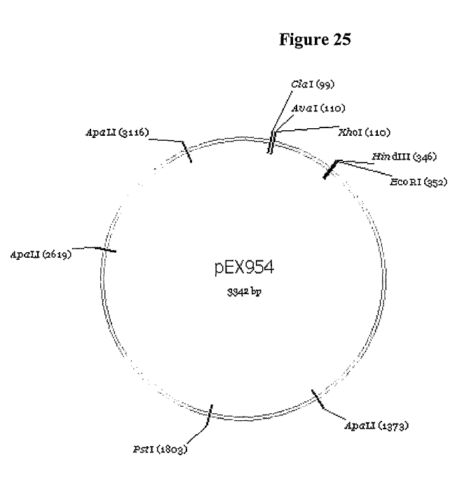

FIG. 25 provides the plasmid map of the pEX954 vector

### Example 1

### Production of DNA Encoding Soluble High Affinity A6 (Tax) TCR-Superantigen Fusion Proteins

Synthetic genes comprising the DNA sequence encoding the soluble high affinity A6 (Tax) TCR β chain detailed in FIG. 5a linked via a DNA sequence encoding a peptide linker to the 5′ end of DNA encoding either the wild-type SEA or mutated SEA E120 superantigens detailed in FIGS. 8a and 9a respectively were synthesised.

There are a number of companies that provide a suitable DNA service, such as Geneart (Germany)

FIG. 10a details the DNA sequence of the high affinity variant of the A6 (Tax) TCR β chain extracellular amino acid sequences containing a non-native cysteine involved in the formation of a novel interchain bond linked to the wild-type SEA E superantigen via a Gly-Ser-Gly-Gly-Pro (SEQ ID NO: 2) linker. The introduced cysteine is indicated by shading. The DNA sequence encoding the Gly-Ser-Gly-Gly-Pro linker is underlined.

FIG. 10b details the amino acid sequence of the high affinity variant of the A6 (Tax) TCR β chain extracellular amino acid sequences containing a non-native cysteine codon involved in the formation of a novel interchain bond linked to the wild-type SEA E superantigen via a Gly-Ser-Gly-Gly-Pro linker (SEQ ID NO: 2). The introduced cysteine is indicated by shading. The Gly-Ser-Gly-Gly-Pro linker is underlined.

FIG. 11a details the DNA sequence of the high affinity variant of the A6 (Tax) TCR β chain extracellular amino acid sequences containing a non-native cysteine involved in the formation of a novel interchain bond linked to the SEA E120 superantigen via a Gly-Ser-Gly-Gly-Pro (SEQ ID NO: 2) linker. The introduced cysteine is indicated by shading. The DNA sequence encoding the Gly-Ser-Gly-Gly-Pro linker is underlined.

FIG. 11b details the amino acid sequence of the high affinity variant of the A6 (Tax) TCR β chain extracellular amino acid sequences containing a non-native cysteine codon involved in the formation of a novel interchain bond linked to the SEA E120 superantigen via a Gly-Ser-Gly-Gly-Pro (SEQ ID NO: 2) linker. The introduced cysteine is indicated by shading. The Gly-Ser-Gly-Gly-Pro linker is underlined.

As is known to those skilled in the art a variety of peptide linkers may be suitable to link the TCR β chains to the superantigens. The following are examples linker sequences which may be used for this purpose

### Linker Sequences:

As is known to those skilled in the art a variety of peptide linkers may be suitable to link the TCR β chains to the superantigens. The following are examples linker sequences which may be used for this purpose

ggcggtccg which encodes a Gly-Gly-Pro linker (L1).
ggatccggcggtccg (SEQ ID NO: 1)—which encodes a Gly-Ser-Gly-Gly-Pro (SEQ ID NO: 2) linker (L2) including a BamH1 restriction enzyme site.
ggatccggtgggggcggaagtggaggcagrggtggatccggeggtccg—(SEQ ID NO: 3) which encodes a Gly-Ser-Gly-Gly-Gly-Gly-Ser-Gly-Gly-Ser-Gly-Gly-Ser-Gly-Gly-Pro(SEQ ID NO: 4) linker (L3) including two BamH1 restriction enzyme sites.
cccggg—which encodes a Pro-Gly linker (L4) including a Xma1 restriction enzyme site

It is also possible to cause the association of soluble TCR chains with superantigens as direct fusions. Fusion proteins formed by direct fusion of soluble TCR β chains and the required superantigen are herein given the assignment TCR-L0-superantigens to denote the lack of any linker sequence. Direct TCR-superantigen fusion proteins may include truncations of either the TCR or superantigen part of the fusion protein. For example, the following two types of truncated TCR-superantigen fusions have been formed. “TCR-LM1-superantigen” fusions comprise a soluble TCR β chain truncated to remove the final three amino acids from the C-terminal of the TCR chain fused directly to the N-Terminal of a superantigen. “TCR-LM2-superantigen” fusions comprise a soluble TCR β chain fused directly to a superantigen which has been truncated so as to remove the first three amino acids from the N-Terminal thereof.

One of the above synthetic genes encoding the TCR β chain-linker-superantigen fusion protein was then sub-cloned into the pEX821 plasmid. The DNA sequence and plasmid map for pEX821 are provided by FIG. 14 and FIG. 23 respectively.

A synthetic gene encoding the cc chain of the soluble A6 (Tax) TCR containing a non-native cysteine codon was then independently sub-cloned into the pEX954 plasmid. (See FIGS. 24 and 25 for the DNA sequence and plasmid map of pEX954 respectively)

FIG. 1a details the DNA sequence of this soluble A6 (Tax) TCR α chain.

### Example 2

### Production of DNA Encoding a Soluble High Affinity Telomerase TCR-Superantigen Fusion Protein

Synthetic genes comprising the DNA sequence encoding the soluble high affinity Telomerase TCR, chain detailed in FIG. 6b linked via a DNA sequence encoding a peptide linker to the 5′ end of DNA encoding either the wild-type SEA or mutated SEA E120 superantigens detailed in FIGS. 8a and 9a respectively were synthesised.

There are a number of companies that provide a suitable DNA service, such as Geneart. (Germany)

FIG. 12a—DNA sequence of a high affinity variant of a Telomerase TCR β chain extracellular amino acid sequences containing a non-native cysteine involved in the formation of a novel interchain bond linked to the wild-type SEA E superantigen via a Gly-Ser-Gly-Gly-Pro (SEQ ID NO: 2) linker. The introduced cysteine is indicated by shading. The DNA sequence encoding the Gly-Ser-Gly-Gly-Pro linker is underlined.

FIG. 12b—Amino acid sequence of a high affinity variant of a Telomerase TCR β chain extracellular amino acid sequences containing a non-native cysteine codon involved in the formation of a novel interchain bond linked to the wild-type SEA E superantigen via a Gly-Ser-Gly-Gly-Pro (SEQ ID NO: 2) linker. The introduced cysteine is indicated by shading. The Gly-Ser-Gly-Gly-Pro linker is underlined.

FIG. 13a—DNA sequence of a high affinity variant of a Telomerase TCR β chain extracellular amino acid sequences containing a non-native cysteine involved in the formation of a novel interchain bond linked to the SEA E120 superantigen via a Gly-Ser-Gly-Gly-Pro (SEQ ID NO: 2) linker. The introduced cysteine is indicated by shading. The DNA sequence encoding the Gly-Ser-Gly-Gly-Pro linker is underlined.

FIG. 13b—Amino acid sequence of a high affinity variant of a Telomerase TCR β chain extracellular amino acid sequences containing a non-native cysteine codon involved in the formation of a novel interchain bond linked to the SEA E120 superantigen via a Gly-Ser-Gly-Gly-Pro (SEQ ID NO: 2) linker. The introduced cysteine is indicated by shading. The Gly-Ser-Gly-Gly-Pro linker is underlined.

As is known to those skilled in the art a variety of peptide linkers may be suitable to link the TCR β chains to the superantigens. Example 1 provides examples of linker sequences which may be used for this purpose

One of the above synthetic genes encoding the TCR β chain-linker-superantigen fusion protein was then sub-cloned into the pEX821 plasmid. FIG. 14 details the DNA sequence of the pEX821 plasmid and FIG. 23 provides the plasmid map for this vector.

A synthetic gene encoding the cc chain of the soluble Telomerase TCR containing a non-native cysteine codon was then independently sub-cloned into the pEX954 plasmid. (See FIGS. 24 and 25 for the DNA sequence and plasmid map of pEX954 respectively)

FIG. 6a details the DNA sequence of this soluble Telomerase TCR cc chain.

As will be obvious to those skilled in the art the methods described in Examples 1 and 2 may be used to produce soluble TCR-superantigen fusion proteins of the invention from any TCR for which the DNA sequence is known. For example, TCR-superantigen fusion proteins containing the soluble Flu-HLA-A2 specific JM22 TCR detailed in FIGS. 3 and 4 can be produced following these methods.

### Example 3

### Expression, Refolding and Purification of Soluble TCR-Superantigen Fusion Proteins

The pEX954 and pEX821 expression plasmids containing the mutated TCR α-chain and TCR β-chain—superantigen fusion proteins respectively were transformed separately into E. coli strain BL21pLysS, and single ampicillin-resistant colonies were grown at 37° C. in TYP (ampicillin 100 μg/ml) medium to OD₆₀₀ of 0.4 before inducing protein expression with 0.5 mM IPTG. Cells were harvested three hours post-induction by centrifugation for 30 minutes at 400 rpm in a Beckman J-6B. Cell pellets were re-suspended in a buffer containing 50 mM Tris-HCl, 25% (w/v) sucrose, 1 mM NaEDTA, 0.1% (w/v) NaAzide, 10 mM DTT, pH 8.0. After an overnight freeze-thaw step, re-suspended cells were sonicated in 1 minute bursts for a total of around 10 minutes in a Milsonix XL2020 sonicator using a standard 12 mm diameter probe. Inclusion body pellets were recovered by centrifugation for 30 minutes at 13000 rpm in a Beckman J2-21 centrifuge. Three detergent washes were then carried out to remove cell debris and membrane components. Each time the inclusion body pellet was homogenised in a Triton buffer (50 mM Tris-HCl, 0.5% Triton-X100, 200 mM NaCl, 10 mM NaEDTA, 0.1% (w/v) NaAzide, 2 mM DTT, pH 8.0) before being pelleted by centrifugation for 15 minutes at 13000 rpm in a Beckman J2-21. Detergent and salt was then removed by a similar wash in the following buffer: 50 mM Tris-HCl, 1 mM NaEDTA, 0.1% (w/v) NaAzide, 2 mM DTT, pH 8.0. Finally, the inclusion bodies were divided into 30 mg aliquots and frozen at −70° C. Inclusion body protein yield was quantitated by solubilising with 6M guanidine-HCl and measurement with a Bradford dye-binding assay (PerBio).

Denaturation of soluble polypeptides; 30 mg of the solubilised TCR β-chain-superantigen inclusion body and 60 mg of the solubilised TCR α-chain inclusion body was thawed from frozen stocks. The inclusion bodies were diluted to a final concentration of 5 mg/ml in 6M guanidine solution, and DTT (2M stock) was added to a final concentration of 10 mM. The mixture was incubated at 37° C. for 30 min. Refolding of soluble TCR-superantigen fusion proteins: 1 L refolding buffer was stirred vigorously at 5° C.±3° C. The redox couple (2-mercaptoethylamine and cystamine (to final concentrations of 6.6 mM and 3.7 mM, respectively) were added approximately 5 minutes before addition of the denatured TCR/TCR-superantigen polypeptides. The protein was then allowed to refold for approximately 5 hours A 15 minutes with stirring at 5° C.±3° C.

Dialysis of refolded soluble TCR-superantigen fusion proteins: The refolded TCR-superantigen fusion proteins was dialysed in Spectrapor 1 membrane (Spectrum; Product No. 132670) against 10 L 10 mM Tris pH 8.1 at 5° C.±3° C. for 18-20 hours. After this time, the dialysis buffer was changed to fresh 10 mM Tris pH 8.1 (10 L) and dialysis was continued at 5° C.±3° C. for another 20-22 hours.

### Example 4

### BIAcore Surface Plasmon Resonance Characterisation of the Binding of TCR-Superantigen Fusion Proteins to Specific pMHC

A surface plasmon resonance biosensor (BIAcore 3000™) was used to analyse the binding of TCR-superantigen fusion proteins to their cognate peptide-MHC ligands. This was facilitated by producing single pMHC complexes (described below) which were immobilised to a streptavidin-coated binding surface in a semi-oriented fashion, allowing efficient testing of the binding of a TCR-superantigen fusion protein to up to four different pMHC (immobilised on separate flow cells) simultaneously. Manual injection of HLA complex allows the precise level of immobilised class I molecules to be manipulated easily.

Such immobilised complexes are capable of binding both T-cell receptors and the co-receptor CD8αα, both of which may be injected in the soluble phase. Specific binding of TCR-superantigen fusion proteins is obtained even at low concentrations (at least 40 μg/ml), implying the TCR-superantigen fusion proteins are relatively stable.

Biotinylated class I HLA-A2-peptide complexes were refolded in vitro from bacterially-expressed inclusion bodies containing the constituent subunit proteins and synthetic peptide, followed by purification and in vitro enzymatic biotinylation (O'Callaghan et al. (1999) Anal. Biochem. 266: 9-15). HLA-heavy chain was expressed with a C-terminal biotinylation tag which replaces the transmembrane and cytoplasmic domains of the protein in an appropriate construct. Inclusion body expression levels of ˜75 mg/litre bacterial culture were obtained. The HLA light-chain or β2-microglobulin was also expressed as inclusion bodies in E. coli from an appropriate construct, at a level of ˜500 mg/litre bacterial culture.

E. coli cells were lysed and inclusion bodies are purified to approximately 80% purity. Protein from inclusion bodies was denatured in 6 M guanidine-HCl, 50 mM Tris pH 8.1, 100 mM NaCl, 10 mM DTT, 10 mM EDTA, and was refolded at a concentration of 30 mg/litre heavy chain, 30 mg/litre β2m into 0.4 M L-Arginine-HCl, 100 mM Tris pH 8.1, 3.7 mM cystamine, mM cysteamine, 4 mg/ml peptide (e.g. Tax 11-19), by addition of a single pulse of denatured protein into refold buffer at <5° C. Refolding was allowed to reach completion at 4° C. for at least 1 hour.

Buffer was exchanged by dialysis in 10 volumes of 10 mM Tris pH 8.1. Two changes of buffer were necessary to reduce the ionic strength of the solution sufficiently. The protein solution was then filtered through a 1.5 μm cellulose acetate filter and loaded onto a POROS 50HQ anion exchange column (8 ml bed volume). Protein was eluted with a linear 0-500 mM NaCl gradient. HLA-A2-peptide complex eluted at approximately 250 mM NaCl, and peak fractions were collected, a cocktail of protease inhibitors (Calbiochem) was added and the fractions were chilled on ice.

Biotinylation tagged HLA complexes were buffer exchanged into 10 mM Tris pH 8.1, 5 mM NaCl using a Pharmacia fast desalting column equilibrated in the same buffer. Immediately upon elution, the protein-containing fractions were chilled on ice and protease inhibitor cocktail (Calbiochem) was added. Biotinylation reagents were then added: 1 mM biotin, 5 mM ATP (buffered to pH 8), 7.5 mM MgCl₂, and 5 μg/ml BirA enzyme (purified according to O'Callaghan et al. (1999) Anal. Biochem. 266: 9-15). The mixture was then allowed to incubate at room temperature overnight.

Biotinylated HLA complexes were purified using gel filtration chromatography. A Pharmacia Superdex 75 HR 10/30 column was pre-equilibrated with filtered PBS and 1 ml of the biotinylation reaction mixture was loaded and the column was developed with PBS at 0.5 ml/min. Biotinylated HLA complexes eluted as a single peak at approximately 15 ml. Fractions containing protein were pooled, chilled on ice, and protease inhibitor cocktail was added. Protein concentration was determined using a Coomassie-binding assay (PerBio) and aliquots of biotinylated HLA complexes were stored frozen at −20° C. Streptavidin was immobilised by standard amine coupling methods.

The interactions between TCR-superantigen fusion proteins and their cognate MHC complex or an irrelevant HLA-peptide combination, the production of which is described above, were analysed on a BLAcore 3000™ surface plasmon resonance (SPR) biosensor. SPR measures changes in refractive index expressed in response units (RU) near a sensor surface within a small flow cell, a principle that can be used to detect receptor ligand interactions and to analyse their affinity and kinetic parameters. The probe flow cells were prepared by immobilising the individual HLA-peptide complexes in separate flow cells via binding between the biotin cross linked onto β2m and streptavidin which have been chemically cross linked to the activated surface of the flow cells. The assay was then performed by passing TCR-superantigen fusion proteins over the surfaces of the different flow cells at a constant flow rate, measuring the SPR response in doing so. Initially, the specificity of the interaction was verified by passing TCR-superantigen fusion proteins at a constant flow rate of 5 μl min-1 over two different surfaces; one coated with ˜5000 RU of specific peptide-HLA complex, the second coated with ˜5000 RU of non-specific peptide-HLA complex. Injections of soluble TCR-superantigen fusion proteins at constant flow rate and different concentrations over the peptide-HLA complex were used to define the background resonance. The values of these control measurements were subtracted from the values obtained with specific peptide-HLA complex and used to calculate binding affinities expressed as the dissociation constant, Kd (Price & Dwek, Principles and Problems in Physical Chemistry for Biochemists (2^{nd} Edition) 1979, Clarendon Press, Oxford).

### Example 5

### In-Vitro Cellular Assay of High Affinity TCR-SEA E-120 Fusion Protein-Mediated Cell Lysis

### Effector Cell Production

On day 0, 2×10⁷ PBMCs isolated from buffy coats in 8 ml of R10 media in a 6-well plate (Nunc) were stimulated with 10 ng/ml wild-type SEA and 25 μl IL-7 (2 μg/ml). The cultures were then incubated at 37° C., 5% CO₂. On day 4, 20 Units/ml IL-2 was added to these cultures.

On day 7, the SEA-stimulated cells were re-stimulated with 1×10⁶ irradiated J82 cancer cells transfected with a mini-gene encoding the Tax peptide (LLFGPVYV) (SEQ ID NO: 7). 1 μg/ml high affinity Tax TCR-SEA E-120 and 20 Units/ml IL-2 were also added to the culture at this time-point. On day 11, 20 Units/ml IL-2 was added to these cultures.

### In-Vitro Cell Lysis Assay

On day 14, 5000 target cells/well (PP-LCL cells, an EBV transformed B-cell line) in 50 μl of R10 media (pulsed with 1×10⁻⁶ M Tax peptide or 1×10⁻⁶ M of an irrelevant Flu peptide for Control wells, and 3 μl BATDA reagent/1×10⁶ cells as directed by the instructions supplied with the Europium/DELFIA assay kit (Perkin Elmer)) were placed in a 96 well plate (Nunc). Further control wells were also prepared that additionally contained 10 μg/ml of an anti-MHC Class II antibody.

The following was then added to the above target cell cultures:

2.25×10⁵ Effector cells prepared as described above in 50 μl of R10 media. (To give an Effector:Target ratio of 45:1)

A range of concentrations (2×10⁻¹¹ M to 2×10⁻⁶ M) of high affinity Tax TCR-SEA E-120 fusion protein in 501 of R10 media.

These cultures were then incubated for 2 hours at 37° C., 5% CO₂. 20 μl of supernatant was then removed from each well and placed into a black opaque 96 well plate (Nunc). 200 μl of Europium solution from the Europium/DELFIA assay kit was then added to each well and the level of target cell lysis that had occurred was assayed by time-resolved fluorescence in a Wallac Victor 2. (Perkin Elmer)

### Calculations:

% cell lysis=100×(RFU_{Exp}−RFU_{Spont})/(RFU_{Max}−RFU_{Spont})

Wherein:

RFU—is relative fluorescence units
RFU_{Exp}—is the RFU measured in the sample wells—cell free background RFU.
RFU_{Spont}—is the RFU measured in the sample wells not containing any Effector cells—cell free background RFU.
RFU_{Max}—is the RFU measured in the sample wells to which triton x-100 was added—cell free background RFU.

### Results

High affinity Tax TCR-SEA E120 fusion-mediated specific killing was demonstrated with an EC50 of 0.2-0.3 nM and maximal killing (almost 100%) at 2 nM. (See FIG. 15)

### Example 6

### Further In-Vitro Cellular Assay of High Affinity Tax TCR-SEA E-120 Fusion Protein-Mediated Cell Lysis

### Effector Cell Production

On day 0, 2×10⁷ PBMCs isolated from buffy coats pulsed with cognate peptide (1×10⁻⁵ M) in 8 ml of R10 media in a 6-well plate (Nunc) were stimulated with 1 μg/ml Tax TCR-L2-SEA E-120 and 25 μl IL-7 (2 μg/ml). The cultures were then incubated at 37° C., 5% CO₂. On day 3, 20 Units/ml IL-2 was added to these cultures.

On day 7, the Tax TCR-L2-SEA-E120 stimulated cells were re-stimulated with 3×10⁶ irradiated J82 cancer cells transfected with a mini-gene encoding the Tax peptide (LLFGPVYV) (SEQ ID NO: 7). 1 g/ml high affinity Tax TCR-SEA E-120 and 20 Units/ml IL-2 were also added to the culture at this time-point. On day 11, 20 Units/ml IL-2 was added to these cultures.

### In-Vitro Cell Lysis Assay

On day 14, 2500 target cells 1 well (T2 cells, an T-B Cell hybridoma line) in 50 μl of R10 media (pulsed with a range (1×10⁻⁶ to 1×10⁻¹⁰M cognate peptide or 1×10⁻⁶ M of an irrelevant Flu peptide for Control wells, and 3 μl BATDA reagent/1×10⁶ cells as directed by the instructions supplied with the Europium/DELFIA assay kit (Perkin Elmer)) were placed in a 96 well plate (Nunc).

The following was then added to the above target cell cultures:

5×10⁴ Effector cells prepared as described above in 50 μl of R10 media. (To give an Effector:Target ratio of 20:1)

A range of concentrations (2×10⁻¹¹ M to 2×10⁻⁶ M) of high affinity TCR-SEA E-120 fusion protein in 501 of R10 media.

These cultures were then incubated for 2 hours at 37° C., 5% CO₂. 20 μl of supernatant was then removed from each well and placed into a black opaque 96 well plate (Nunc). 200 μl of Europium solution from the Europium/DELFIA assay kit was then added to each well and the level of target cell lysis that had occurred was assayed by time-resolved fluorescence in a Wallac Victor 2. (Perkin Elmer)

### Calculations:

As described in Example 5 above.

### Results

An initial experiment was carried out using T2 target cells to investigate the effect of titrating Tax peptide pulsing concentration (1×10⁻¹⁰ to 1×10⁻⁶M) and high affinity Tax TCR-L2-SEA E-120 concentration (6.4×10⁻¹³ to 1×10⁻⁷M). A maximum of 80-90% cell lysis was obtained. (See FIG. 16)

The above experiment was repeated using a fixed concentration (10⁻⁶M) of Tax peptide or an irrelevant Telomerase peptide (ILAKFLHWL) control. A maximum of 80-90% cell lysis was obtained using the cognate Tax peptide, compared to approximately 20% cell lysis using the irrelevant peptide. (See FIG. 17)

The cell killing ability of a number of TCR-superantigen fusions (Tax TCR-L2-SEA E120, Telomerase TCR-L1-SEA E120 and NY-ESO TCR-LM1-SEA E120) all supplied at a concentration of 2 nM was tested against T2 target cells pulsed with 10⁻⁶M cognate or irrelevant peptide at a rate of Effector:Target (E:T) cell ratios using the assay described above.

The DNA and amino acid sequence of the high affinity NY-ESO TCR cc chain containing an introduced cysteine are detailed in FIGS. 18a and 18b respectively. The DNA and amino acid sequence of the truncated high affinity NY-ESO TCR β chain containing an introduced cysteine fused to SEA E-120 are detailed in FIGS. 19a and 19b respectively. The truncated NY-ESO TCR β chain has the last 3 amino acids (RAD) removed from the C-Terminal thereof compared to the “normal” soluble TCR. The codon encoding the final glycine residue in the DNA sequence of the truncated TCR β chain is underlined in FIG. 19a, as is the corresponding glycine residue in FIG. 19b.

The maximum lysis obtained was approximately 50%, 75% and 100% for the Tax TCR-L2-SEA E120, Telomerase TCR-L1-SEA E120 and NY-ESO TCR-LM1-SEA E120 fusion proteins respectively. (See FIGS. 20a-20c respectively)

The cell killing ability of two different TCR-superantigen fusions (Tax TCR-L2-SEA E120 and Telomerase TCR-L1-SEA E120) both supplied at a concentration of 2 nM was tested against T2 target cells pulsed with 10⁻⁶M of the cognate peptide for one of these TCR-superantigen fusions using the assay described above at a range of Effector:Target (E:T) cell ratios. The maximum lysis obtained was approximately 65% and 75% for the Tax TCR-L2-SEA E120 and Telomerase TCR-L1-SEA E120 fusion proteins respectively. (See FIGS. 21a and 21b respectively)

The cell killing ability of the Tax TCR-L2-SEA E120 and Telomerase TCR-L1-SEA E120 fusion proteins both supplied at a concentration of 2 nM was tested against SK-Mel 37 tumour cell line target cells pulsed with 10⁻⁶M of Tax peptide using the assay described above at a range of Effector:Target (E:T) cell ratios. The maximum lysis obtained was approximately 35-40% using the cognate Tax TCR-L2-SEA E120 fusion protein, compared to approximately 10% for the Telomerase TCR-L1-SEA E120 fusion protein. (See FIG. 22)

1. A soluble bifunctional protein comprising an association between
(a) a T cell receptor (TCR) and
(b) a superantigen.

2. A soluble bifunctional protein as claimed in claim 1, wherein component (a) is a TCR, which comprises (i) all or part of a TCR chain, except the transmembrane domain thereof, and (ii) all or part of a TCR β chain, except the transmembrane domain thereof, wherein (i) and (ii) each comprise a functional variable domain and at least a part of the constant domain of the TCR chain, and are linked by a disulfide bond between constant domain residues which is not present in native TCR.

3. A soluble bifunctional protein as claimed in claim 2, wherein the N-terminus of the superantigen is covalently linked directly, or indirectly via a linker radical, to the C-terminal amino acid of the said TCR α chain or TCR β chain.

4. A soluble bifunctional protein as claimed in claim 2, wherein the N-terminus of the superantigen is:
directly linked to the C-terminal amino acid of the said TCR α chain or TCR β chain via a peptide bond, or inter cysteine disulfide bond, or
linked indirectly via a peptide bond, or inter cysteine disulfide bond, to the C-terminal amino acid of a linker amino acid sequence which is itself linked via a peptide bond, or inter cysteine disulfide bond, to the C-terminus of the said TCR α chain or TCR β chain.

5. A soluble bifunctional protein as claimed in claim 2, wherein the N-terminus of the superantigen is linked to the C-terminal amino acid of the said TCR α chain or TCR β chain via a non-peptidic polymeric radical.

6. A soluble bifunctional protein as claimed in claim 1, wherein the superantigen is a mutant of a wild type superantigen, wherein the mutation reduces the affinity of the superantigen for Class II MHC molecules whilst retaining the affinity for TCR β variable domains.

7. A soluble bifunctional protein as claimed in claim 1, wherein the superantigen is a wild type or mutated staphylococcal superantigen.

8. A soluble bifunctional protein as claimed in claim 1, wherein the superantigen has the amino acid sequence SEQ ID NO: 5.

9. A soluble bifunctional protein as claimed in claim 2, wherein, in the TCR part (i) and (ii) comprise all of the extracellular constant Ig domain of the TCR chain.

10. A soluble bifunctional protein as claimed in claim 2, wherein, in the TCR part (i) and (ii) comprises all of the extracellular domain of the TCR chain.

11. A soluble bifunctional protein as claimed in claim 2, wherein, in the TCR part, a covalent disulfide bond links a residue of the immunoglobulin region of the constant domain of the α chain to a residue of the immunoglobulin region of the constant domain of the β chain.

12. A soluble bifunctional protein as claimed in claim 1, wherein, in the TCR part, an interchain disulfide bond present in native TCRs is absent.

13. A soluble bifunctional protein as claimed in claim 12, wherein, in the TCR part, native α and β TCR chains are truncated at the C-terminus such that the cysteine residues which form the native interchain disulfide bond are excluded.

14. A soluble bifunctional protein as claimed in claim 12, wherein, in the TCR part, cysteine residues which form the native interchain disulfide bond are substituted to another residue.

15. A soluble bifunctional protein as claimed in claim 14, wherein, in the TCR part, cysteine residues which form the native interchain disulfide bond are substituted to serine or alanine.

16. A soluble bifunctional protein as claimed in claim 1, wherein, in the TCR part, an unpaired cysteine residue present in native TCR β chains is not present.

17. A soluble bifunctional protein as claimed in claim 2, wherein, in the TCR part, the disulfide bond which is not present in native TCRs is between cysteine residues substituted for residues whose β carbon atoms are less than 0.6 nm apart in the native TCR structure.

18. A soluble bifunctional protein as claimed in claim 2, wherein, in the TCR part, the disulfide bond which is not present in native TCRs is between cysteine residues substituted for Thr 48 of exon 1 of TRAC*01 and Ser 57 of exon 1 of TRBC1*01 or TRBC2*01.

19. A soluble bifunctional protein as claimed in claim 2, comprising an association between:
(a) a T cell receptor (TCR), which comprises (i) all or part of a TCR α chain, except the transmembrane domain thereof, and (ii) all or part of a TCR β chain, except the transmembrane domain thereof, wherein (i) and (ii) each comprise a functional variable domain and at least a part of the constant domain of the TCR chain, and are linked by a disulfide bond between cysteine residues substituted for Thr 48 of exon 1 of TRAC*01 and Ser 57 of exon 1 of TRBC1*01 or TRBC2*01, and;
(b) the superantigen of SEQ ID NO: 5,
wherein the association TCR (a) and the superantigen (b) are associated in C-terminal to N-terminal relationship respectively.

20. A soluble bifunctional protein as claimed in claim 1, further comprising a detectable label.

21. A soluble bifunctional protein as claimed in claim 1, wherein the TCR has an affinity (Kd) for a given peptide-MHC of higher than 1 μM.

22. A multivalent complex comprising a plurality of soluble bifunctional proteins as claimed in claim 1.

23. A complex as claimed in claim 22, comprising two or three or four associated bifunctional proteins associated with one another via a linker radical comprising a polyalkylene glycol polymer or a peptidic sequence.

24. A method for enriching a diverse population of T cells for T cells presenting a given sub-set of TCR β chain variable domains, which comprises:
(i) providing a soluble bifunctional protein as claimed in claim 1 or a multivalent complex thereof wherein the superantigen part thereof selectively binds to said given sub-set of TCR β chain variable domains;
(ii) contacting the soluble bifunctional protein or multivalent complex with antigen presenting cells (APCs) presenting Class II MHC-peptide complexes, and said diverse population of T cells; and
(iii) incubating the admixture of (ii) under conditions suitable for the formation of APC-soluble bifunctional protein-T cell complexes.

25. A pharmaceutical formulation comprising a soluble bifunctional protein as claimed in claim 1, and/or a multivalent complex thereof together with a pharmaceutically acceptable carrier.

26. A nucleic acid molecule comprising a first nucleic acid sequence encoding a superantigen fused to a second nucleic acid encoding a soluble TCR β chain.

27. A nucleic acid molecule comprising a first nucleic acid sequence encoding a superantigen fused to a second nucleic acid encoding all or part of a TCR β chain except the transmembrane domain thereof, wherein the nucleic acid sequence encoding the TCR β chain comprises an introduced cysteine codon capable of forming a non-native disulfide bond between the constant domain residues of the encoded TCR β chain and a TCR α chain containing a further non-native cysteine residue.

28. A nucleic acid molecule comprising a first nucleic acid sequence encoding a superantigen fused to a second nucleic acid encoding a soluble TCR α chain.

29. A nucleic acid molecule comprising a first nucleic acid sequence encoding a superantigen fused to a second nucleic acid encoding all or part of a TCR α chain except the transmembrane domain thereof, wherein the nucleic acid sequence encoding the TCR α chain comprises an introduced cysteine codon capable of forming a non-native disulfide bond between the constant domain residues of the encoded TCR α chain and a TCR β chain containing a further non-native cysteine residue.

30. A vector comprising a nucleic acid molecule or molecules as claimed in claim 26.

31. A host cell comprising a vector as claimed in claim 30.

32. A method for obtaining a soluble bifunctional protein, which method comprises:
incubating a host cell which comprises a vector comprising a nucleic acid molecule encoding a TCR β chain fused to a superantigen and a host cell which comprises a vector comprising a nucleic acid molecule encoding a TCR α chain under conditions causing expression of the respective TCR β chain-superantigen fusion and TCR α chain;
purifying the respective TCR β chain-superantigen fusion and TCR α chain; and
mixing the respective TCR β chain-superantigen fusion and TCR α chain under refolding conditions such that a covalent disulfide bond links a residue of the immunoglobulin region of the constant domain of the TCR α chain to a residue of the immunoglobulin region of the constant domain of the TCR β chain-superantigen fusion.

33. A method for obtaining a soluble bifunctional protein, which method comprises:
incubating a host cell which comprises a vector comprising a nucleic acid molecule encoding a TCR α chain fused to a superantigen and a host cell which comprises a vector comprising a nucleic acid molecule encoding a TCR β chain under conditions causing expression of the respective TCR α chain-superantigen fusion and TCR β chain;
purifying the respective TCR α chain-superantigen fusion and TCR β chain; and
mixing the respective TCR α chain-superantigen fusion and TCR β chain under refolding conditions such that a covalent disulfide bond links a residue of the immunoglobulin region of the constant domain of the TCR β chain to a residue of the immunoglobulin region of the constant domain of the TCR α chain-superantigen fusion.

34. A method of treatment of cancer comprising administering to a subject suffering such cancer an effective amount of a soluble bifunctional protein or a functional variant or fragment thereof as claimed in claim 1 or a multivalent complex thereof.

35. A method of treatment of cancer comprising administering to a subject suffering such cancer an effective amount of a soluble bifunctional protein as claimed in 8, or a multivalent complex thereof.

36. (canceled)

37. (canceled)

38. A method of treatment of infectious disease comprising administering to a subject suffering such infectious disease an effective amount of a soluble bifunctional protein or a functional variant or fragment thereof as claimed in claim 1 or a multivalent complex thereof.

39. A method of treatment of infectious disease comprising administering to a subject suffering such infectious disease an effective amount of a soluble bifunctional protein or a functional variant or fragment thereof as claimed in claim 8 or a multivalent complex thereof.

40. (canceled)

41. (canceled)


SEQUENCE LISTING (37 sequences)

SEQ ID NO: 1 - DNA, 15 bp, Artificial Sequence
  DNA encoding L2 linker
ggatccggcggtccg

SEQ ID NO: 2 - protein, 5 aa, Artificial Sequence
  L2 Linker
GSGGP

SEQ ID NO: 3 - DNA, 48 bp, Artificial Sequence
  DNA encoding L3 Linker
ggatccggtgggggcggaagtggaggcagcggtggatccggcggtccg

SEQ ID NO: 4 - protein, 16 aa, Artificial Sequence
  L3 Linker
GSGGGGSGGSGGSGGP

SEQ ID NO: 5 - protein, 234 aa, Artificial Sequence
  Mutated SEA-E120 Superantigen
MSEKSEEINEKDLRKKSELQGTALGNLKQIYYYNSKAITSSEKSADQFLTNTLLFKGFFTGHPWYNDLLVDLGSTAATSEYEGSSVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNTTISSTSLSISLYLYTT

SEQ ID NO: 6 - protein, 233 aa, Staphylococcus aureus
SEKSEEINEKDLRKKSELQGTALGNLKQIYYYNEKAKTENKESHDQFLQHTILFKGFFTDHSWYNDLLVDFDSKDIVDKYKGKKVDLYGAYYGYQCAGGTPNKTACMYGGVTLHDNNRLTEEKKVPINLWLDGKQNTVPLETVKTNKKNVTVQELDLQARRYLQEKYNLYNSDVFDGKVQRGLIVFHTSTEPSVNYDLFGAQGQYSNTLLRIYRDNKTINSENMHIDIYLYTS

SEQ ID NO: 7 - protein, 8 aa, Artificial Sequence
  Tax peptide
LLFGPVYV

SEQ ID NO: 8 - DNA, 621 bp, Artificial Sequence
  DN encoding a soluble DiS linked A6 (Tax) TCR alpha chain
atgcagaaggaagtggagcagaactctggacccctcagtgttccagagggagccattgcctctctcaactgcacttacagtgaccgaggttcccagtccttcttctggtacagacaatattctgggaaaagccctgagttgataatgtccatatactccaatggtgacaaagaagatggaaggtttacagcacagctcaataaagccagccagtatgtttctctgctcatcagagactcccagcccagtgattcagccacctacctctgtgccgttacaactgacagctgggggaaattgcagtttggagcagggacccaggttgtggtcaccccagatatccagaacccggatcctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctaa

SEQ ID NO: 9 - DNA, 741 bp, Artificial Sequence
  DNA encoding a soluble DiS linked A6 (Tax) TCR alpha chain
atgaacgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcaggccgggactagcgggagggcgaccagagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagactaa

SEQ ID NO: 10 - protein, 206 aa, Artificial Sequence
  Amino acid sequence of a soluble DiS linked A6 (Tax) TCR alpha chain
MQKEVEQNSGPLSVPEGAIASLNCTYSDRGSQSFFWYRQYSGKSPELIMSIYSNGDKEDGRFTAQLNKASQYVSLLIRDSQPSDSATYLCAVTTDSWGKLQFGAGTQVVVTPDIQNPDPAVYQLRDSKSSDKSVCLFTDFDSQTNVSQSKDSDVYITDKCVLDMRSMDFKSNSAVAWSNKSDFACANAFNNSIIPEDTFFPSPESS

SEQ ID NO: 11 - protein, 246 aa, Artificial Sequence
  Amino acid sequence of a soluble DiS linked A6 (Tax) TCR beta chain
MNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASRPGLAGGRPEQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 12 - DNA, 615 bp, Artificial Sequence
  DNA encoding a soluble DiS linked JM22 TCR alpha chain
atgcaactactagaacaaagtcctcagtttctaagcatccaagagggagaaaatctcactgtgtactgcaactcctcaagtgttttttccagcttacaatggtacagacaggagcctggggaaggtcctgtcctcctggtgacagtagttacgggtggagaagtgaagaagctgaagagactaacctttcagtttggtgatgcaagaaaggacagttctctccacatcactgcggcccagcctggtgatacaggcctctacctctgtgcaggagcgggaagccaaggaaatctcatctttggaaaaggcactaaactctctgttaaaccaaatatccagaacccggatcctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctaa

SEQ ID NO: 13 - DNA, 735 bp, Artificial Sequence
  DNA encoding a soluble DiS linked JM22 TCR beta chain
atggtggatggtggaatcactcagtccccaaagtacctgttcagaaaggaaggacagaatgtgaccctgagttgtgaacagaatttgaaccacgatgccatgtactggtaccgacaggacccagggcaagggctgagattgatctactactcacagatagtaaatgactttcagaaaggagatatagctgaagggtacagcgtctctcgggagaagaaggaatcctttcctctcactgtgacatcggcccaaaagaacccgacagctttctatctctgtgccagtagttcgaggagctcctacgagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatacagcctgagcagccgcctgagggtctcggccaccttctggcagaacccccgcaaccacttccgctgtcaagtccagttctacgggctctcggagaatgacgagtggacccaggatagggccaaacctgtcacccagattgtcagcgccgaggcctggggtagagcagactaa

SEQ ID NO: 14 - protein, 204 aa, Artificial Sequence
  Amino acid sequence of a soluble DiS linked JM22TCR alpha chain
MQLLEQSPQFLSIQEGENLTVYCNSSSVFSSLQWYRQEPGEGPVLLVTVVTGGEVKKLKRLTFQFGDARKDSSLHITAAQPGDTGLYLCAGAGSQGNLIFGKGTKLSVKPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESS

SEQ ID NO: 15 - protein, 244 aa, Artificial Sequence
  Amino acid sequence of a soluble DiS linked JM22TCR beta chain
MVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSSRSSYEQYFGPGTRLTVTEDLKNVFPPEVAVFEPSEAEISHTQKATLVCLATGFYPDHVELSWWVNGKEVHSGVCTDPQPLKEQPALNDSRYSLSSRLRVSATFWQNPRNHFRCQVQFYGLSENDEWTQDRAKPVTQIVSAEAWGRAD

SEQ ID NO: 16 - DNA, 741 bp, Artificial Sequence
  DNA encoding a soluble DiS linked high affinity A6 TCR beta
atgaacgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgcctcgaggccggggctgatgagtgcggaaccagagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccaccggtttctaccccgaccacgtggagctgagctggtgggtgaatgggaaggaggtgcacagtggggtctgcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagactaa

SEQ ID NO: 17 - protein, 246 aa, Artificial Sequence
  Amino acid sequence of a soluble DiS linked high affinity A6 TCR beta chain
MNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASRPGLMSAQPEQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 18 - DNA, 621 bp, Artificial Sequence
  DNA encoding a soluble DiS linked high affinity Telomerase TCR alpha chain
atgggaatacaagtggagcagagtcctccagacctgattctccaggagggagccaattccacgctgcggtgcaatttttctgactctgtgaacaatttgcagtggtttcatcaaaacccttggggacagctcatcaacctgttttacattccctcagggacaaaacagaatggaagattaagcgccacgactgtcgctacggaacgctacagcttattgtacatttcctcttcccagaccacagactcaggcgtttatttctgtgctgtggactctgctaccgcgctgccgtatgggtacatctttggaacaggcaccaggctgaaggttttagcaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctaa

SEQ ID NO: 19 - DNA, 729 bp, Artificial Sequence
  DNA encoding a soluble DiS linked high affinity Telomerase TCR beta chain
atgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgggattcagcctcagggcactgaagctttctttggacaaggcaccagactcacagttgtagaggacctgaacaaggtgttcccacccgaggtcgctgtgtttgagccatcagaagcagagatctcccacacccaaaaggccacactggtgtgcctggccaccggtttctaccccgaccacgtggagctgagctggtgggtgaatgggaaggaggtgcacagtggggtctgcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagactaa

SEQ ID NO: 20 - protein, 206 aa, Artificial Sequence
  Amino acid sequence of a soluble DiS linked high affinity Telomerase TCR alpha chain
MGIQVEQSPPDLILQEGANSTLRCNFSDSVNNLQWFHQNPWGQLINLFYIPSGTKQNGRLSATTVATERYSLLYISSSQTTDSGVYFCAVDSATALPYGYIFGTGTRLKVLANIQNPDPAVYQLRDSKSSDKSVCLFTDFDSQTNVSQSKDSDVYITDKCVLDMRSMDFKSNSAVAWSNKSDFACANAFNNSIIPEDTFFPSPESS

SEQ ID NO: 21 - protein, 242 aa, Artificial Sequence
  Amino acid sequence of a soluble DiS linked high affinity Telomerase TCR beta chain
MNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCGIQPQGTEAFFGQGTRLTVVEDLNKVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 22 - DNA, 702 bp, Staphylococcus aureus
agcgagaaaagcgaagaaataaatgaaaaagatttgcgaaaaaagtctgaattgcagggaacagctttaggcaatcttaaacaaatctattattacaatgaaaaagctaaaactgaaaataaagagagtcacgatcaatttttacagcatactatattgtttaaaggcttttttacagatcattcgtggtataacgatttattagtagattttgattcaaaggatattgttgataaatataaagggaaaaaagtagacttgtatggtgcttattatggttatcaatgtgcgggtggtacaccaaacaaaacagcttgtatgtatggtggtgtaacgttacatgataataatcgattgaccgaagagaaaaaagtgccgatcaatttatggctagacggtaaacaaaatacagtacctttggaaacggttaaaacgaataagaaaaatgtaactgttcaggagttggatcttcaagcaagacgttatttacaggaaaaatataatttatataactctgatgtttttgatgggaaggttcagaggggattaatcgtgtttcatacttctacagaaccttcggttaattacgatttatttggtgctcaaggacagtattcaaatacactattaagaatatatagagataataaaacgattaactctgaaaacatgcatattgatatatatttatatacaagttaa

SEQ ID NO: 23 - DNA, 705 bp, Artificial Sequence
  DNA encoding a mutated SEA-E120 superantigen
atgtccgagaagagcgaagaaataaatgaaaaagatctgcgaaaaaagtctgagctccaaggtaccgccctaggcaatcttaagcaaatttattattataatagcaaagctataactagcagcgaaaagagtgcggatcagtttctcacgaatactttgttatttaaaggttttttcacaggtcatccatggtataacgacttactagtggatcttggttcaaccgcggctactagcgaatatgaagggagtagtgtagatctatatggtgcttattatggatatcaatgtgctggaggcacaccaaataaaacagcatgtatgtacgggggtgtaacattacatgataataatcgattgaccgaagaaaaaaaagtaccaattaacttgtggatagacggaaaacaaactacagtacctatagataaagttaaaacaagcaaaaaagaagtaactgttcaagagctagaccttcaggcgcgccattatttacacggaaaatttggtttatataactcagacagctttggcggtaaggtgcaaagaggcttgattgtgtttcattcttctgaagggtccacggtaagttatgatttgtttgatgctcaagggcaatatccagatacattactccggatttacagagataataccactatttcgtctacgagcctcagcattagcttgtatttgtacacaacttaa

SEQ ID NO: 24 - DNA, 1455 bp, Artificial Sequence
  DNA encoding a soluble high affinity A6 (Tax) TCR beta chain - L2 linker - WT SEA-E fusion protein
atgaacgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgcctcgaggccggggctgatgagtgcggaaccagagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccaccggtttctaccccgaccacgtggagctgagctggtgggtgaatgggaaggaggtgcacagtggggtctgcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagacggatccggcggtccgagcgagaaaagcgaagaaataaatgaaaaagatttgcgaaaaaagtctgaattgcagggaacagctttaggcaatcttaaacaaatctattattacaatgaaaaagctaaaactgaaaataaagagagtcacgatcaatttttacagcatactatattgtttaaaggcttttttacagatcattcgtggtataacgatttattagtagattttgattcaaaggatattgttgataaatataaagggaaaaaagtagacttgtatggtgcttattatggttatcaatgtgcgggtggtacaccaaacaaaacagcttgtatgtatggtggtgtaacgttacatgataataatcgattgaccgaagagaaaaaagtgccgatcaatttatggctagacggtaaacaaaatacagtacctttggaaacggttaaaacgaataagaaaaatgtaactgttcaggagttggatcttcaagcaagacgttatttacaggaaaaatataatttatataactctgatgtttttgatgggaaggttcagaggggattaatcgtgtttcatacttctacagaaccttcggttaattacgatttatttggtgctcaaggacagtattcaaatacactattaagaatatatagagataataaaacgattaactctgaaaacatgcatattgatatatatttatatacaagttaa

SEQ ID NO: 25 - protein, 486 aa, Artificial Sequence
  Amino acid sequence of a soluble high affinity A6 (Tax) TCR beta chain - L2 linker - WT SEA-E fusion protein
MNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASRPGLMSAQPEQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRADGSGGPSEKSEEINEKDLRKKSELQGTALGNLKQIYYYNEKAKTENKESHDQFLQHTILFKGFFTDHSWYNDLLVDFDSKDIVDKYKGKKVDLYGAYYGYQCAGGTPNKTACMYGGVTLHDNNRLTEEKKVPINLWLDGKQNTVPLETVKTNKKNVTVQELDLQARRYLQEKYNLYNSDVFDGKVQRGLIVFHTSTEPSVNYDLFGAQGQYSNTLLRIYRDNKTINSENMHIDIYLYTSAI

SEQ ID NO: 26 - DNA, 1455 bp, Artificial Sequence
  DNA encoding a soluble high affinity A6 (Tax) TCR beta chain - L2 linker - SEA-E120 fusion protein
atgaacgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgcctcgaggccggggctgatgagtgcggaaccagagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccaccggtttctaccccgaccacgtggagctgagctggtgggtgaatgggaaggaggtgcacagtggggtctgcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagacggatccggcggtccgtccgagaagagcgaagaaataaatgaaaaagatctgcgaaaaaagtctgagctccaaggtaccgccctaggcaatcttaagcaaatttattattataatagcaaagctataactagcagcgaaaagagtgcggatcagtttctcacgaatactttgttatttaaaggttttttcacaggtcatccatggtataacgacttactagtggatcttggttcaaccgcggctactagcgaatatgaagggagtagtgtagatctatatggtgcttattatggatatcaatgtgctggaggcacaccaaataaaacagcatgtatgtacgggggtgtaacattacatgataataatcgattgaccgaagaaaaaaaagtaccaattaacttgtggatagacggaaaacaaactacagtacctatagataaagttaaaacaagcaaaaaagaagtaactgttcaagagctagaccttcaggcgcgccattatttacacggaaaatttggtttatataactcagacagctttggcggtaaggtgcaaagaggcttgattgtgtttcattcttctgaagggtccacggtaagttatgatttgtttgatgctcaagggcaatatccagatacattactccggatttacagagataataccactatttcgtctacgagcctcagcattagcttgtatttgtacacaacttaa

SEQ ID NO: 27 - protein, 484 aa, Artificial Sequence
  Amino acid sequence of a soluble high affinity A6 (Tax) TCR beta chain - L2 linker - SEA-E120 fusion protein
MNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASRPGLMSAQPEQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRADGSGGPSEKSEEINEKDLRKKSELQGTALGNLKQIYYYNSKAITSSEKSADQFLTNTLLFKGFFTGHPWYNDLLVDLGSTAATSEYEGSSVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNTTISSTSLSISLYLYTT

SEQ ID NO: 28 - DNA, 1443 bp, Artificial Sequence
  DNA encoding a soluble high affinity Telomerase TCR beta chain - L2 linker - WT SEA-E fusion protein
atgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgggattcagcctcagggcactgaagctttctttggacaaggcaccagactcacagttgtagaggacctgaacaaggtgttcccacccgaggtcgctgtgtttgagccatcagaagcagagatctcccacacccaaaaggccacactggtgtgcctggccaccggtttctaccccgaccacgtggagctgagctggtgggtgaatgggaaggaggtgcacagtggggtctgcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagacggatccggcggtccgagcgagaaaagcgaagaaataaatgaaaaagatttgcgaaaaaagtctgaattgcagggaacagctttaggcaatcttaaacaaatctattattacaatgaaaaagctaaaactgaaaataaagagagtcacgatcaatttttacagcatactatattgtttaaaggcttttttacagatcattcgtggtataacgatttattagtagattttgattcaaaggatattgttgataaatataaagggaaaaaagtagacttgtatggtgcttattatggttatcaatgtgcgggtggtacaccaaacaaaacagcttgtatgtatggtggtgtaacgttacatgataataatcgattgaccgaagagaaaaaagtgccgatcaatttatggctagacggtaaacaaaatacagtacctttggaaacggttaaaacgaataagaaaaatgtaactgttcaggagttggatcttcaagcaagacgttatttacaggaaaaatataatttatataactctgatgtttttgatgggaaggttcagaggggattaatcgtgtttcatacttctacagaaccttcggttaattacgatttatttggtgctcaaggacagtattcaaatacactattaagaatatatagagataataaaacgattaactctgaaaacatgcatattgatatatatttatatacaagttaa

SEQ ID NO: 29 - protein, 479 aa, Artificial Sequence
  Amino acid sequence of a soluble high affinity Telomerase TCR beta chain - L2 linker - WT SEA-E fusion protein
MNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCGIQPQGTEAFFGQGTRLTVVEDLNKVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRADGSGGPSEKSEEINEKDLRKKSELQGTALGNLKQIYYYNEKAKTENKESHDQFLQHTILFKGFFTDHSWYNDLLVDFDSKDIVDKYKGKKVDLYGAYYGYQCAGGTPNKTACMYGGVTLHDNNRLTEEKKVPINLWLDGKQNTVPLETVKTNKKNVTVQELDLQARRYLQEKYNLYNSDVFDGKVQRGLIVFHTSTEPSVNYDLFGAQGQYSNTLLRIYRDNKTINSENMHIDIYLYT

SEQ ID NO: 30 - DNA, 1443 bp, Artificial Sequence
  DNA encoding a soluble high affinity Telomerase TCR beta chain - L2 linker - SEA-E120 fusion protein
atgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgggattcagcctcagggcactgaagctttctttggacaaggcaccagactcacagttgtagaggacctgaacaaggtgttcccacccgaggtcgctgtgtttgagccatcagaagcagagatctcccacacccaaaaggccacactggtgtgcctggccaccggtttctaccccgaccacgtggagctgagctggtgggtgaatgggaaggaggtgcacagtggggtctgcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagacggatccggcggtccgtccgagaagagcgaagaaataaatgaaaaagatctgcgaaaaaagtctgagctccaaggtaccgccctaggcaatcttaagcaaatttattattataatagcaaagctataactagcagcgaaaagagtgcggatcagtttctcacgaatactttgttatttaaaggttttttcacaggtcatccatggtataacgacttactagtggatcttggttcaaccgcggctactagcgaatatgaagggagtagtgtagatctatatggtgcttattatggatatcaatgtgctggaggcacaccaaataaaacagcatgtatgtacgggggtgtaacattacatgataataatcgattgaccgaagaaaaaaaagtaccaattaacttgtggatagacggaaaacaaactacagtacctatagataaagttaaaacaagcaaaaaagaagtaactgttcaagagctagaccttcaggcgcgccattatttacacggaaaatttggtttatataactcagacagctttggcggtaaggtgcaaagaggcttgattgtgtttcattcttctgaagggtccacggtaagttatgatttgtttgatgctcaagggcaatatccagatacattactccggatttacagagataataccactatttcgtctacgagcctcagcattagcttgtatttgtacacaacttaa

SEQ ID NO: 31 - protein, 480 aa, Artificial Sequence
  Amino acid sequence of a soluble high affinity Telomerase TCR beta chain - L2 linker - SEA-E120 fusion protein
MNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCGIQPQGTEAFFGQGTRLTVVEDLNKVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRADGSGGPSEKSEEINEKDLRKKSELQGTALGNLKQIYYYNSKAITSSEKSADQFLTNTLLFKGFFTGHPWYNDLLVDLGSTAATSEYEGSSVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNTTISSTSLSISLYLYTT

SEQ ID NO: 32 - DNA, 3836 bp, Artificial Sequence
  DNA sequence of the pEX821 plasmid
gatctcgatcccgcgaaattaatacgactcactatagggagaccacaacggtttccctctagaaataattttgtttaactttaagaaggagatatacatatgaacgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcaggccgggactagcgggagggcgaccagagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccaccggtttctaccccgaccacgtggagctgagctggtgggtgaatgggaaggaggtgcacagtggggtctgcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagactaagcttgaattccgatccggctgctaacaaagcccgaaaggaagctgagttggctgctgccaccgctgagcaataactagcataaccccttggggcctctaaacgggtcttgaggggttttttgctgaaaggaggaactatatccggataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcag

SEQ ID NO: 33 - DNA, 630 bp, Artificial Sequence
  DNA encoding a soluble DiS linked high affinity NY-ESO TCR alpha chain
atgaaacaggaggtgacgcagattcctgcagctctgagtgtcccagaaggagaaaacttggttctcaactgcagtttcactgatagcgctatttacaacctccagtggtttaggcaggaccctgggaaaggtctcacatctctgttgcttattaccccgtggcagagagagcaaacaagtggaagacttaatgcctcgctggataaatcatcaggacgtagtactttatacattgcagcttctcagcctggtgactcagccacctacctctgtgctgtgaggccccttttggatggtacttacatacctacatttggaagaggaaccagccttattgttcatccgtatatccagaacccggatcctgccgtgtaccagctgagagactctaagtcgagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctaa

SEQ ID NO: 34 - protein, 208 aa, Artificial Sequence
  Amino acid sequence of a soluble DiS linked high affinity NY-ESO TCR alpha chain
MQEVTQIPAALSVPEGENLVLNCSFTDSAIYNLQWFRQDPGKGLTSLLLITPWQREQTSGRLNASLDKSSGRSTLYIAASQPGDSATYLCAVRPLLDGSYIPTFGRGTSLIVHPYIQNPDPAVYQLRDSKSSDKSVCLFTDFDSQTNVSQSKDSDVYITDKCVLDMRSMDFKSNSAVAWSNKSDFACANAFNNSIIPEDTFFPSPESS

SEQ ID NO: 35 - DNA, 1425 bp, Artificial Sequence
  DNA encoding a truncated soluble DiS linked high affinity NY-ESO TCR beta chain fused to SEA E120
atgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttgctattcagaccactgaccaaggagaagtccccaatggctacaatgtctccagatcaaccatagaggatttcccgctcaggctgctgtcggctgctccctcccagacatctgtgtacttctgtgccagcagttacctcgggaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccaccggtttctaccccgaccacgtggagctgagctggtgggtgaatgggaaggaggtgcacagtggggtctgcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctgggggtccgagaagagcgaagaaataaatgaaaaagatctgcgaaaaaagtctgagctccaaggtaccgccctaggcaatcttaagcaaatttattattataatagcaaagctataactagcagcgaaaagagtgcggatcagtttctcacgaatactttgttatttaaaggttttttcacaggtcatccatggtataacgacttactagtggatcttggttcaaccgcggctactagcgaatatgaagggagtagtgtagatctatatggtgcttattatggatatcaatgtgctggaggcacaccaaataaaacagcatgtatgtacgggggtgtaacattacatgataataatcgattgaccgaagaaaaaaaagtaccaattaacttgtggatagacggaaaacaaactacagtacctatagataaagttaaaacaagcaaaaaagaagtaactgttcaagagctagaccttcaggcgcgccattatttacacggaaaatttggtttatataactcagacagctttggcggtaaggtgcaaagaggcttgattgtgtttcattcttctgaagggtccacggtaagttatgatttgtttgatgctcaagggcaatatccagatacattactccggatttacagagataataccactatttcgtctacgagcctcagcattagcttgtatttgtacacaacttaa

SEQ ID NO: 36 - protein, 472 aa, Artificial Sequence
  Amino acid sequence of a truncated soluble DiS linked high affinity NY-ESO TCR beta chain fused to SEA E120
MGVTQTPKFQVLKTGQSMTLQCAQDMNHEYMSWYRQDPGMGLRLIHYSVAIQTTDRGEVPNGYNVSRSTIEDFPLRLLSAAPSQTSVYFCASSYLGNTGELFFGEGSRLTVLEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGSEKSEEINEKDLRKKSELQGTALGNLKQIYYYNSKAITSSEKSADQFLTNTLLFKGFFTGHPWYNDLLVDLGSTAATSEYEGSSVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNTTISSTSLSISLYLYTT

SEQ ID NO: 37 - DNA, 3342 bp, Artificial Sequence
  pEX954 vector
gatctcgatcccgcgaaattaatacgactcactatagggagaccacaacggtttccctctagaaataattttgtttaactttaagaaggagatataatcgatgtctaactcgagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctaagcttgaattccgatccggctgctaacaaagcccgaaaggaagctgagttggctgctgccaccgctgagcaataactagcataaccccttggggcctctaaacgggtcttgaggggttttttgctgaaaggaggaactatatccggataattcttgaagacgaaagggcctcgtgatacgcctatttttataggttaatgtcatgataataatggtttcttagacgtgaggtggcacttttcggggaaatgtgcgcggaacccctatttgtttatttttctaaatacattcaaatatgtatccgctcatgagacaataaccctgataaatgcttcaataatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcag